SEQUENCE LISTING (8 sequences)

SEQ ID NO: 1 - DNA, 1050 bp, Homo sapiens
  CDS (32)..(748)
gttggcccccgttacttttcctctgggaaatatggcgcacgctgggagaacagggtacgataaccgggagatagtgatgaagtacatccattataagctgtcgcagaggggctacgagtgggatgcgggagatgtgggcgccgcgcccccgggggccgcccccgcgccgggcatcttctcctcgcagcccgggcacacgccccatacagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccaggcagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgttccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatc

SEQ ID NO: 2 - protein, 239 aa, Homo sapiens
MAHAGRTGYDNREIVMKYIHYKLSQRGYEWDAGDVGAAPPGAAPAPGIFSSQPGHTPHTAASRDPVARTSPLQTPAAPGAAAGPALSPVPPVVHLTLRQAGDDFSRRYRRDFAEMSRQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 3 - DNA, 926 bp, Homo sapiens
  CDS (135)..(833)
gaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaacccg

SEQ ID NO: 4 - protein, 233 aa, Homo sapiens [sequence omitted]

SEQ ID NO: 5 - DNA, 20 bp, Artificial Sequence
  Description of Artificial Sequence Primer
gctgcagacagactggccag

SEQ ID NO: 6 - DNA, 20 bp, Artificial Sequence
  Description of Artificial Sequence Primer
aggcatcgcgcacatccagc

SEQ ID NO: 7 - DNA, 19 bp, Artificial Sequence
  Description of Artificial Sequence Primer
ctggaagccggcgcagatc

SEQ ID NO: 8 - DNA, 20 bp, Artificial Sequence
  Description of Artificial Sequence Primer
gcgtgtccaggaagccttcc

(19) United States
(12) Patent Application Publication
(10) Pub. No.: US 2003/0103945 A1
(43) Pub. Date: Jun. 5, 2003

(54) Methods and compositions for stimulating axon regeneration and preventing neuronal cell degeneration

(76) Inventors: Dong Feng Chen, Newton, MA (US); Xizhong Huang, Framingham, MA (US); Guang Chen, Potomac, MD (US); Husseini K. Manji, Rockville, MD (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/072,830

(22) Filed: Feb. 8, 2002

Related U.S. Application Data
(60) Provisional application No. 60/267,832, filed on Feb. 9, 2001.
(60) Provisional application No. 60/272,617, filed on Mar. 1, 2001.
(60) Provisional application No. 60/289,990, filed on May 10, 2001.

(51) Int. Cl. A61K 33/00; A61K 38/18; C12N 5/08

(52) U.S. Cl. 424/93.7; 424/722; 514/12; 435/368

(57) ABSTRACT

Agents which modulate a bcl family member to control axonal growth and regeneration are described. These bcl modulating agents promote axonal growth and regeneration in the neural cells of a subject. Compositions for promoting axonal cell growth in a subject also are described. The compositions of the present invention include an effective amount of an agent which modulates a bcl family member and in a pharmaceutically acceptable carrier. Other described aspects include packaged drugs for treating a state characterized by diminished potential for axonal growth. The packaged compounds and agents also include instructions for using the agent to promote axonal growth in a subject. An exemplary agent is lithium or a salt thereof.

### CROSS-REFERENCE TO RELATED APPLICATIONS

This application claims the benefit of U.S. Provisional Application No. 60/267,832, filed Feb. 9, 2001; U.S. Provisional Application No. 60/272,617, filed Mar. 1, 2001; and U.S. Provisional Application No. 60/289,990, filed May 10, 2001, the contents of which are specifically incorporated by reference herein.

### STATEMENT OF RIGHTS

This invention was made during the course of work supported by NIH grant EY012983.

### BACKGROUND OF THE INVENTION

The functions of the brain and spinal cord depend on cells called neurons, which contact and communicate with each other through nerve fibers called axons. Injuries to the brain or spinal cord can cause the loss of many axons and the disruption of connections between neurons in the brain and spinal cord. This disruption results in the devastating loss of function in patients with such injuries, leaving them with varying degrees of paralysis and losses in sensory or cognitive functions. Some of these losses are permanent since there is very little regeneration of these axons in mammals.

Most neurons of the mammalian central nervous system (CNS) lose the ability to regenerate severed axons after a certain point in development (Aubert, I., et al. Curr. Opin. Biol. 5, 625-635 (1995); Baehr, M. & Bonhoeffer, F. TINS 17, 473-479 (1994)). Acutely damaged CNS neurons do, however, make an abortive attempt at regenerating. It has been suggested that axotomized neurons in the CNS are able to produce new axons, as in the peripheral nervous system (PNS), but that regeneration fails because of the non-permissive nature of the environment in which the new growth cones are formed (Breckness and Fawcett. Biol. Rev. 71:227 (1996)). Early work suggested that the nonpermissive CNS environment resulted from the lack of chemical factors which were present in the PNS (Cajal. Degeneration and Regeneration of the Nervous System, Oxford University Press, Oxford (1928)). Among the molecules thought to be important in axonal regeneration are the neurotrophins, which include: nerve growth factor (NGF), brain-derived neurotrophic factor (BDNF), neurotrophin-3 (NT-3), NT-4/5, and NT-6 (Silos-Santiago et al. Curr. Opin. Neurobiol. 5:42 (1995); Davies. TINS 18:355(1995)). The receptors of the Trk family are thought to play key roles in the mechanism of action of neurotrophins (Greene and Kaplan. Curr. Opinion in NeurobioL 5:579 (1995)). Other non-neurotrophin growth factors are thought to influence neuronal populations, including: ciliary neurotrophic factor (CNTF), leukemia inhibitory factor (LIF), insulin-like growth factor (IGF)-I and IGF-II, glial cell line derived neurotrophic factor (GDNF), growth promoting activity (GPA), basic fibroblast growth factor (bFGF) and members of the transforming growth factor β (TGFB) superfamily (Silos-Santiago et al.; Davies supra). Apolipoprotein E, and laminin are also thought to play a role in axonal regeneration (Breckness and Fawcett, supra). The mature CNS, however, is not devoid of all of these factors. Another explanation for the failure of axonal regeneration in the CNS has been that the CNS contains inhibitors of axonal growth, such as proteins found in the membranes of oligodendrocytes and CNS myelin (Schnell, L. & Schwab, M. E. Nature 343, 269-272 (1990)). More recent evidence, however, indicates that the ability of embryonic neurons to develop axons may be a property of the neurons themselves. For example, embryonic neurons are better at growing axons than adult neurons are at regenerating them, even when those embryonic neurons are placed in an adult CNS environment.

Embryonic neurons transplanted into the adult CNS are able to form long axons, even along myelinated tracts (Wictorin et al., Nature 347:556 (1990); Davies et al. Journal of Neurosciences 14:1596(1994)).

One protein which has been implicated in axonal growth is GAP-43. A correlation has been found between the expression of GAP-43 (also known as B-50, pp46, neuromodulin, and F 1) and the ability of a neural cell to regenerate an axon. GAP-43 is a phosphoprotein found in neuronal growth cones, which has been found to bind to calmodulin (Spencer and Willard. Exp. Neurol. 115:167 (1991)) and to stimulate nucleoside triphosphate binding to the G protein, Go (Strittmatter et al. Nature 344:836 (1990)). While the relationship between the synthesis of GAP-43 and periods of axon extension, has suggested its role in axonal growth (Fidel et al. Neurosci, Abstr. 16:339(1990); Schotman et al., Soc. Neruosci. Abstr. 16:339(1990)), some axotomized RGCs have been shown to up-regulate GAP-43 without regenerating (Doster et al. Neuron 6:635(1991); Schaden et al., Journal of Neurobiology 25:1570(1994)). Moreover, PC 12 cells have been shown to extend neurites in the absence of GAP-43 (Baetge and Hammang Neuron 6:21(1991)).

The bcl-2 gene was discovered at the breakpoint region of the t(14; 18) chromosomal translocation. Bcl-2 is a 26 kD integral membrane protein that has been localized to the outer mitochondrial membrane, perinuclear membrane and smooth endoplasmic reticulum, and has been shown to be important in the regulation of apoptosis (Nunez et al. Immunology Today 15:583 (1994)). Apoptosis is also known as “programmed cell death” and involves the activation in cells of a genetic program leading to cell death. Apoptosis occurs in both normal cell development and certain disease states. For example, downregulation of bcl-2 is a common feature of normal lymphoid populations undergoing programmed cell death and selection, whereas upregulation of bcl-2 appears to be part of the positive selection mechanism (Nunez et al. supra). The death of neurons which occurs in Alzheimer's dementia and Parkinson's disease, as well as in cancer and viral infection, also shows the hallmarks of apoptosis.

It would be highly desirable to have methods for regenerating axons on neurons for treating neuronal diseases, e.g., CNS degeneration that involves cell loss and nerve damage.

### SUMMARY OF THE INVENTION

The present invention is based, at least in part, on the discovery that bcl-2 plays a role in the growth and/or regeneration of axons in neural cells. The present invention pertains to compositions and methods of promoting axonal growth in a neural cell. The method involves modulating the expression or bioactivity of a bcl family member in a neural cell such that axonal growth occurs. The invention further pertains to methods of treating a subject for a state characterized by diminished potential for axonal growth. The method involves administering a therapeutically effective amount of an agent which modulates the bioactivity or expression of a bcl family member in a subject such that axonal growth occurs. In one embodiment, the agent is a gene construct for expressing a bcl family member. The gene construct is formulated for delivery into neural cells of the subject such that axonal growth occurs. The agent can also be a bcl family member polypeptide. In yet another embodiment, the agent increases the expression of bcl2, e.g., lithium or an analog thereof. The invention also provides methods for preventing neural cell degeneration essentially without stimulating axonal growth. Such methods include contacting a neural cell with a bclx_{L} gene, polypeptide or agent that stimulates its expression or bioactivity. Other aspects of the invention include pharmaceutical preparations and packaged drugs used in the aforementioned methods. Methods for selecting agents or bcl family members for use within the aforementioned methods also are part of this invention.

The invention further provides compositions comprising lithium or a salt thereof and an agent that creates an environment favorable for axonal growth and a pharmaceutically acceptable carrier. The agent can be selected from the group consisting of NGF, BDNF, NT-3, 4, 5, or 6, CNTF, LIF, IGFI, IGFII, GDNF, GPA, bFGF, TGFB, and apolipoprotein E. Compositions can be in a vehicle for administration to a subject, such as a tube, catheter, syringe or stent. The compositions can also be in the form of a tablet.

The invention also provides methods for promoting axonal growth in a neural cell, comprising contacting the neural cell with an amount of lithium or salt thereof sufficient to stimulate axonal growth, such that axonal growth occurs. The neural cell can be, e.g., a central nervous system (CNS) cell or a peripheral nervous system.

In another embodiment, the invention provides methods for treating a subject that has suffered a traumatic injury in which nerve cell injury has occurred, comprising administering to the subject lithium or a salt thereof, in an amount sufficient to stimulate axon regeneration, such that the subject is treated. Administering may comprise providing lithium or a salt thereof to the site of nerve cell injury, such as by injection. An agent that creates an environment favorable for axonal growth can further be administered, such at the site of the nerve cell injury. The nerve cell injury can be a spinal cord injury or a peripheral nervous system injury. The nerve cell injury can be an optic nerve injury.

Also provided are methods for treating a subject for a state characterized by diminished potential axonal growth, comprising administering to the subject lithium or a salt thereof, in an amount sufficient to stimulate axonal growth, such that the subject is treated. The state can be a CNS disorder; a peripheral nervous system disorder or an opitic, e.g., retinal injury or degeneration, e.g., glaucoma.

In other embodiments, the invention provides methods for stimulating axon growth of a neural cell in vitro, comprising contacting a neural cell with an amount of lithium or salt thereof sufficient to stimulate axon growth, such that the neural cell growths at least one axon. The neural cell can be obtained from a subject. The neural cell can also be a cell that was differentiated from a stem cell. The cell with at least one exon can be administered to a subject, e.g., a subject from which the neural cell was obtained.

In another embodiment, the invention provides methods for preventing neural cell degeneration, comprising contacting the neural cell with an agent that increases the amount of Bcl-x_{L} in the neural cell, such that neural cell degeneration is prevented. The neural cell can be contacted with a nucleic acid encoding a Bcl-x_{L} protein or portion thereof sufficient for preventing neural cell degeneration. The neural cell can also be contacted with a Bcl-x_{L} protein, such that the protein enters the neural cell. In another embodiment, the invention provides methods for treating a neurodegenerative disease in a subject, comprising contacting neural cells of the subject that are undergoing neurodegeneration with an agent that increases the amount of Bcl-x_{L} in the neural cell, such that the neurodegenerative disease is treated in the subject.

In another embodiment, the invention provides methods for treating a subject having a partial or complete sectioning of the spinal cord or a nerve, comprising (i) providing the ends of the spinal cord or nerve within less than about 100 μm distance from each other; and (ii) contacting at least one cell from the spinal cord or nerve with an agent that increases the level of bcl-2 protein within the cell, such that the cell grows at least one axon, to thereby treat the subject. The agent can be provided at the site of the sectioning of the spinal cord or nerve. The agent can be lithium or a salt thereof.

### BRIEF DESCRIPTION OF THE FIGURES

FIG. 1 shows that the expression of bcl-2 is essential for the growth of most retinal axons in culture: Retinal axon growth was quantitated in cultures from wild-type (C57BL/6J), bcl-2 null mice, and bcl-2 transgenic mice. (A) Quantification of cultures derived from embryonic day 15 pups genetically deficient in bcl-2: retinal explant derived from heterozygous (+/−) or homozygous (−/−) mutant mice both showed decreased numbers of axons that invaded the tectal tissue when compared with those of wild-type animals (+/+) at this age. (B) Growth of retinal axons from adult retinae was quantitated. Retinal explants derived from adult transgenic mice display 10-fold more axonal growth into E16 tectum than into comparable tissues from wild type mice. (C) Growth curves of retinal axons obtained from retinotectal cocultures, using tissues from wild-type or transgenic animals aged embryonic day 14 through day 5 after birth. Mouse genotype was determined by genomic Southern or PCR analysis of genomic DNA isolated from the mouse tails. Data obtained from wild-type mice are plotted with the solid line, and those from transgenic mice are depicted by the dotted line. Note that at age E 18 or older, there is a marked decrease in numbers of retinal axons from wild type animals. This decline was not observed for bcl-2 transgenic mice.

FIG. 2 shows that ZVAD (Z-Val-Ala-Asp-CH₂F, Enzyme Systems Products), though sufficient to prevent death of RGCs, is not sufficient to promote axonal growth: This figure shows the effects of the ICE-like protease inhibitor, ZVAD, on the survival and neurite outgrowth of RGCs in culture. (A) Shows the numbers of surviving RGCs in dissociated retinal cell cultures treated with different doses of ZVAD. Doses from 0 to 200 M were tested. (B) Shows the quantification of cell death in retinal explants from ZVAD-treated retinotectal cocultures. Three doses of ZVAD (50, 100, and 200M) were examined, and cultures were prepared from 2 day old wild-type animals. (C) Quantification of retinal axon growth in coculture experiments parallel to those in (B). Note that by increasing the concentration of ZVAD, the number of dying cells in retinal explants decreased, whereas, the number of growing axons did not change significantly.

FIG. 3 shows the measurements of the distance of optic nerve elongation in Bcl-2-overexpressing mice at 1, 2, and 4 DPO. Values are presented as mean±S.D.

FIG. 4 is a bar chart representing the number of retrogradely labeled RGCs in non-crushed control and/or optic nerve injured retinas of wild-type and Bcl-2-overexpressing mice.

Values are presented as mean±S.D.

FIG. 5 shows the number of TUNEL-positive cells in retinal sections of wild-type, Bcl-x_{L}- and Bcl-2-overexpressing mice at 1 DPO. Values are presented as mean±S.D.

FIG. 6 shows the number of retinal axon regeneration in co-cultures prepared from wild-type mice, mice overexpressing Bcl-x_{L} and Bcl-2. Values are presented as mean±S.D.

FIG. 7A is a bar graph showing a dose response curve of the number of labeled axons invading the tectal slice as a function of increasing amounts of LiCl in retino-tectal co-culture experiments. All data represent mean±S. D. from at least three independent experiments (*p<0.05).

FIG. 7B is a bar graph showing a dose response curve of the longest distances of labeled axons that crossed the retino-tectal border and extended into the tectal slice as a function of increasing amounts of LiCl in retino-tectal co-culture experiments. All data represent mean±S. D. from at least three independent experiments (*p<0.05).

FIG. 8 is a bar graph representing the quantitative data of surviving RGCs in the absence and the presence of LiCl after 5 days in culture. Error bars indicated S.D. (*p<0.05)

FIG. 9 is a bar graph indicating the amount of bcl2 in retinas treated with LiCl. Band intensities were read and analyzed with NIH image program and normalized to the levels of G3PDH obtained from the same preparation. Each data correspond to the ratio of the normalized band intensity.

FIG. 10A is a bar graph showing average number of retinal axon regenerated in retino-1 tectal co-cultures prepared from tissues of wild-type (WT), Bcl-2 heterozygous (+/−), and Bcl-2 homozygous (−/−) knockout mice and maintained in the absence or the presence of LiCl (1 mM). Error bars indicated S.D. (*p<0.05).

FIG. 10B is a bar graph showing the average length of retinal axon regenerated in retino-tectal co-cultures prepared from tissues of wild-type (WT), Bcl-2 heterozygous (+/−), and Bcl-2 homozygous (−/−) knockout mice and maintained in the absence or the presence of LiCl (1 mM). Error bars indicated S.D. (*p<0.05).

FIG. 11A is a bar graph showing average numbers of axons recorded from the retinal-tectal co-culture experiments, in which retinal and tectal tissues were taken from neonatal wild-type mice or mice carrying bcl-2 transgenes, and the cultures were maintained in the absence or the presence of LiCl (1 mM). Error bars indicated S.D. (*p<0.05).

FIG. 11B is a bar graph showing average lengths of axons recorded from the retinal-tectal co-culture experiments, in which retinal and tectal tissues were taken from neonatal wild-type mice or mice carrying bcl-2 transgenes, and the cultures were maintained in the absence or the presence of LiCl (1 mM). Error bars indicated S.D. (*p<0.05).

### DETAILED DESCRIPTION OF THE INVENTION

The present invention provides for methods of promoting axonal growth in a neural cell.

The methods involve modulating the expression or bioactivity of a bcl family member. As used herein, the term “axonal growth” refers to the ability of a bcl modulating agent to enhance the extension (e.g., regeneration) of axons and/or the reestablishment of nerve cell connectivity. Axonal growth as used herein is not intended to include within its scope all neurite sprouting nor is it intended to include the promotion of neural cell survival through means other than the promotion of axonal growth. For example, axonal growth is intended to include neurite sprouting which occurs after an axon is damaged and neurite sprouting which occurs in conjunction with the extension of the axon. Axonal growth as used herein includes axonal regeneration in severed neurons which occurs at, or near, the site at which the axon was severed. The term “neural cell” as used herein is meant to include cells from both the central nervous system (CNS) and the peripheral nervous system (PNS). Exemplary neural cells of the CNS are found in the gray matter of the spinal cord or the brain and exemplary neural cells of the PNS are found in the dorsal root ganglia.

“Neuron,” “neuronal cell” and “neural cell” are used interchangeably to refer to nerve cells, i.e., cells that are responsible for conducting nerve impulses from one part of the body to another. Most neurons consist of three distinct portions: a cell body, soma or perikaryon, which contains a nucleus and two kinds of cytoplasmic processes: dendrites and axons. Dendrites are usually highly branched, thick extensions of the cytoplasm of the cell body. An axon is sually a single long, thin process that is highly specialized and conducts nerve impulses away from the cell body to another neuron or muscular or glandular tissue. Along the length of an axon, there may be side branches called “axon collaterals.” Axon collaterals and axons may terminate by branching into many fine filaments called “axon terminals.” The distal ends of axon terminals are called “synaptic end bulbs,” which contain synaptic vesicles that store neurotransmitters. Axons may be surrounded by a multilayered, white, phospholipid, segmented covering called the myelin sheath. Axons containing such a covering are “myelinated.” Neurons include sensory neurons, which transmit impulses from receptors in the skin, sense organs, muscles, joints, and viscera to the brain and spinal cord and from lower to higher centers of the CNS. A neuron can also be a motor (efferent) neuron convey impulses from the brain and spinal cord to effectors, which may be either muscles or glands, and from higher to lower centers of the CNS. Other neurons are association (connecting or interneuron) neurons which carry impulses from sensory neurons to motor neurons and are located in the brain and spinal cord. Examples of association neurons include stellate cells, cells of Martinotti, horizontal cells of Cajal, pyramidal cells, granule cells and Purkinje cells. The processes of afferent and efferent neurons arranged into bundles called “nerves” when located outside the CNS or fiber tracts if inside the CNS.

“Nerve fiber” refers to an axon and its sheaths. Nerve fibers can be general somatic afferent fibers, general somatic efferent fibers, general visceral afferent fibers and general visceral efferent fibers (“autonomous fibers”).

“White matter” refers to aggregations of myelinated processes from many neurons.

“Gray matter” refers to the part of the nervous system that contains either nerve cell bodies and dendrites or bundles of unmyelinated axons and neuroglia.

The term “bcl family member” or “bcl polypeptide” as used in the instant application is meant to include polypeptides, such as bcl-2 and other members of the bel family. Bcl family member is meant to include within its scope fragments of a bcl family member which possess a bcl bioactivity. Such members can be readily identified using the subject screening assays, described herein. In other embodiments “bcl family members” include polypeptides which comprise bcl domains, which confer bcl bioactivity, such as, for example, BH1, BH2, or BH4.

The terms protein, polypeptide, and peptide are used interchangeably herein. Exemplary bcl family members include: bcl-2, Bcl-xL, Bcl-xs, Bad, Bax, and others (Merry, D. E. et al. 1-5 Development 120:301 (1994); Nifiez, G. et al. Immunol. Today 15, 582-588 (1994)). In preferred embodiments the bcl family member is a bcl-xL molecule or fragment thereof. Human bcl-xL nucleotide and amino acid sequences can be found, e.g., as GenBank no. Z23115, described in Boise et al. (1993) Cell 74:597 (SEQ ID NOs: 3 and 4, respectively). In particularly preferred embodiments the bcl family member is a bcl-2 molecule or fragment thereof. Human bcl-2 nucleotide and amino acid sequences can be found, e.g., as GenBank no. M14745, described in Cleary et al. (1986) Cell 47:19 (SEQ ID NOs: 1 and 2, respectively). Agents that “modulate” the expression or bioactivity of a bcl family member is meant to include agents which either up or downregulate the expression or bioactivity of a bcl family member. In preferred embodiments, a modulating agent upregulates the expression or bioactivity of a bcl family member. Agents which upregulate expression make a quantitative change in the amount of a bcl family member in a cell, while agents which upregulate the bioactivity of a bcl family member make a qualitative change in the ability of a bcl family member to perform a bcl bioactivity. Such agents can be useful therapeutically to promote axonal growth in a cell. Accordingly, the subject methods can be carried out with BCL family member modulating agents described herein, such as, nucleic acids, peptides, and peptidomimetics, or modulating agents identified in drug screens which have a BCL family member bioactivity, for example, which agonize or antagonize the effects of a BCL family member protein. In one aspect of the invention, bcl modulating agents are nucleic acids encoding a bcl family member polypeptide which are introduced into a cell. Exemplary agents are bcl family member nucleic acids, for example in plasmids or viral vectors. As used herein, the term “nucleic acid” refers to polynucleotides such as deoxyribonueleic acid (DNA), and, where appropriate, ribonucleic acid (RNA). The term should also he understood to include, as equivalents, analogs of either RNA or DNA made from nucleotide analogs, and, as applicable to the embodiment being described, single (sense or antisense) and double-stranded polynucleotides.

The use of nucleic acids having a sequence that differs from a bcl family member nucleotide sequences due to degeneracy in the genetic code are also within the scope of the invention. Such nucleic acids encode functionally equivalent peptides (i.e., a peptide having a bioactivity of a bcl polypeptide) but which differ in sequence from the sequence shown in the sequence listing due to degeneracy in the genetic code. It is understood that limited modifications to the protein can be made without destroying the biological function of the bcl family member and that only a portion of the entire primary structure may be required in order to effect activity. For example, a number of amino acids are designated by more than one triplet. Codons that specify the same amino acid, or synonyms (for example, CAU and CAC each encode histidine) may result in “silent” mutations which do not affect the amino acid sequence of a bcl polypeptide. These modifications may be deliberate, such as through site-directed mutagenesis, or accidental, e.g., through mutation. Furthermore, various other modifications can be made to the bcl family member, such as the addition of carbohydrates or lipids. Furthermore, the use of homologous bcl family members, having a bcl bioactivity, from other species is also provided for. As used herein, a bcl modulating agent can also be a nucleic acid encoding a fragment of a bcl polypeptide. A fragment refers to a nucleic acid having fewer nucleotides than the nucleotide sequence encoding the entire mature form of a bcl protein yet which encodes a polypeptide which retains some bioactivity of the full length protein. Thus, fragments of a bcl family member which retain a bcl bioactivity are included with the definition of a bcl family member. In certain embodiments fragments encode a bcl family member polypeptide of at least about 50, at least about 75, or at least about 100 amino acids. In preferred embodiments fragments encode a bcl family of at least about 150 amino acids. In more preferred embodiments fragments encode a bcl family of at least about 200 amino acids. In particularly preferred embodiments fragments encode a bcl family of at least about 239 amino acids.

Bcl protein-encoding nucleic acids can be obtained from mRNA present in any of a number of eukaryotic cells. Nucleic acids encoding bcl polypeptides of the present invention also can be obtained from genomic DNA from both adults and embryos. For example, a gene encoding a bcl protein can be cloned from either a cDNA or a genomic library in accordance with protocols described herein, as well as those generally known to persons skilled in the art. A cDNA encoding a bcl protein can be obtained by isolating total mRNA from a cell, e.g. a mammalian cell, e.g. a human cell, including embryonic cells. Double stranded cDNAs can then be prepared from the total mRNA, and subsequently inserted into a suitable plasmid or bacteriophage vector using any one of a number of known techniques. The gene encoding a bcl protein can also be cloned using established polymerase chain reaction techniques in accordance with the nucleotide sequence information provided by the invention. Alternatively, chemical synthesis of a bcl family member gene sequence can be performed in an automatic DNA synthesizer. The bcl nucleic acid of the invention can be either DNA or RNA.

In another embodiment a modulating agent can be a bcl family member polypeptide which can be administered directly to a neural cell, such as, conjugated to a carrier molecule, e.g., a transcytosis protein. For example, certain small peptides, such as a 9 amino acid region from the HIV TAT protein can be used to efficiently transport peptides from the extracellular milieu into cells. A portion, such as amino acids 42-58, of the Drosophila Antennapedia protein (Ant) can also be used for that effect. Importantly, these peptides can serve as carriers for the introduction of very large molecules, including proteins, into mammalian cells. For example, the HIV TATpeptide can be used.

The polypeptide of this invention can be a full length protein or fragment thereof. The fragment is of a size which allows it to perform its intended function. For example, the family member polypeptide can have a length of at least about 20 amino acids, at least about 50 amino acids, at least about 75 amino acids, at least about 100 amino acids, or at least about 150 amino acids.

In other embodiments, a bcl modulating agent can be a bcl family member which has undergone posttranslational modification. For example, bcl-2 in which a putative negative regulatory loop, containing the major serine/threonine phosphorylation sites, of the protein has been deleted has been shown to have enhanced activity (Galewski and Thompson. 1996. Cell 87:589). BCL family members which are modified to resist proteolysis may also have enhanced activity (Strack et al. 1996. Proc. Natl. Acad. Sci. USA 93:9571).

In certain embodiments it will be advantageous to provide homologs of one of the subject BCL family member polypeptides which function in a limited capacity as one of either a BCL family member agonist (mimetic) or a BCL family member antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of BCL family member proteins.

Homologs of each of the subject BCL family member proteins can be generated by mutagenesis, such as by discrete point mutation(s), or by truncation. For instance, mutation can give rise to homologs which retain substantially the same, or merely a subset, of the biological activity of the BCL family member polypeptide from which it was derived. Alternatively, antagonistic forms of the protein can be generated which are able to inhibit the function of the naturally occurring form of the protein, such as by competitively binding to a BCL family member binding protein. In addition, agonistic forms of the protein may be generated which are constituatively active. Thus, the mammalian BCL family member protein and homologs thereof provided by the subject invention may be either positive or negative regulators of axonal growth.

The recombinant BCL family member polypeptides of the present invention also include homologs of the wild type BCL family member proteins, such as versions of those proteins which are resistant to proteolytic cleavage, as for example, due to mutations which alter ubiquitination or other enzymatic targeting associated with the protein.

BCL family member polypeptides may also be chemically modified to create BCL family member derivatives by forming covalent or aggregate conjugates with other chemical moieties, such as glycosyl groups, lipids, phosphate, acetyl groups and the like. Covalent derivatives of BCL family member proteins can be prepared by linking the chemical moieties to functional groups on amino acid sidechains of the protein or at the N-terminus or at the C-terminus of the polypeptide.

Modification of the structure of the subject mammalian BCL family member polypeptides can be for such purposes as enhancing therapeutic or prophylactic efficacy, stability (e.g., ex vivo shelf life and resistance to proteolytic degradation in vivo), or post-translational modifications (e.g., to alter the phosphorylation pattern of protein). Such modified peptides, when designed to retain at least one activity of the naturally-occurring form of the protein, or to produce specific antagonists thereof, are considered functional equivalents of the BCL family member polypeptides described in more detail herein. Such modified peptides can be produced, for instance, by amino acid substitution, deletion, or addition.

For example, it is reasonable to expect that an isolated replacement of leucine with an isoleucine or valine, an aspartate with a glutamate, a threonine with a serine, or a similar replacement of an amino acid with a structurally related amino acid (i.e. isosteric and/or isoclectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine, histidine, (3) aliphatic=glycine, alanine, valine, leucine, isoleucine, serine, threonine, with serine and threonine optionally grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W H Freeman and Co.: 1981). Whether a change in the amino acid sequence of a peptide results in a functional BCL family member homolog (e.g. functional in the sense that the resulting polypeptide mimics or antagonizes the wild type form) can be readily determined by assessing the ability of the variant peptide to produce a response in cells in a fashion similar to the wild-type protein, or to competitively inhibit such a response. Polypeptides in which more than one replacement has taken place can readily be tested in the same manner.

Full length proteins or fragments corresponding to one or more particular motifs and/or domains or to arbitrary sizes, for example, at least about 5, 10, 25, 50, 75, 100, 125, 150 amino acids in length are within the scope of the present invention. For example, isolated BCL family member polypeptides can include all or a portion of an amino acid sequence corresponding to a BCL family member polypeptide. Isolated peptidyl portions of BCL family member proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t Boc chemistry. For example, a BCL family member polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild type (e.g., “authentic”) BCL family member protein.

This invention further provides a method for generating sets of combinatorial mutants of the subject BCL family member proteins as well as truncation mutants, and is especially useful for identifying potential variant sequences (e.g. homologs) that modulate a BCL family member bioactivity. The purpose of screening such combinatorial libraries is to generate, for example, novel BCL family member homologs which can act as either agonists or antagonist, or alternatively, possess all together novel activities. To illustrate, combinatorially derived homologs can be generated to have an increased potency relative to a naturally occurring form of the protein.

Likewise, BCL family member homologs can be generated by the present combinatorial approach to selectively inhibit (antagonize) an authentic BCL family member. For instance, mutagenesis can provide BCL family member homologs which are able to bind other signal pathway proteins (or DNA) yet prevent propagation of the signal, e.g. the homologs can be dominant negative mutants. Moreover, manipulation of certain domains of BCL family members by the present method can provide domains more suitable for use in fusion proteins.

In one embodiment, the variegated library of BCL family member variants is generated by combinatorial mutagenesis at the nucleic acid level, and is encoded by a variegated gene library. For instance, a mixture of synthetic oligonucleotides can be enzymatically ligated into gene sequences such that the degenerate set of potential BCL family member sequences are expressible as individual polypeptides, or alternatively, as a set of larger fusion proteins (e.g. for phage display) containing the set of BCL family member sequences therein.

There are many ways by which such libraries of potential BCL family member homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential BCL family member sequences. The synthesis of degenerate oligonucleotides is well known in the art (see for example, Narang, SA(1983) Tetrahedron 39:3; Itakuraetal. (1991) Recombinant DNA, Proc 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-1390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for a BCL family member clone in order to generate a variegated population of BCL family member fragments for screening and subsequent selection of bioactive fragments. A variety of techniques are known in the art for generating such libraries, including chemical synthesis. In one embodiment, a library of coding sequence fragments can be generated by (i) treating a double stranded PCR fragment of a BCL family member coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with SI nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of BCL family member homologs. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate BCL family member sequences created by combinatorial mutagenesis techniques.

In one embodiment, cell based assays can be exploited to analyze the variegated BCL family member library. For instance, the library of expression vectors can be transfected into a neural cell line, preferably a neural cell line that does not express a functional BCL family member. The effect of the BCL family member mutant can be detected, e.g. axonal growth. Plasmid DNA can then be recovered from the cells which show potentiation of a BCL family member bioactivity, and the individual clones further characterized.

Combinatorial mutagenesis has the potential to generate very large libraries of mutant proteins, e.g., in the order of 10²⁶ molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the mammalian BCL family member proteins to generate mimetics, e.g. peptide or non-peptide agents. In certain embodiments such mimetics are able to disrupt binding of a mammalian BCL family member polypeptide of the present invention with BCL family members binding proteins or interactors. Thus, such mutagenic techniques as described above are also useful to map the determinants of the BCL family member proteins which participate in protein protein interactions involved in, for example, binding of the subject mammalian BCL family member polypeptide to proteins which may function upstream (including both activators and repressors of its activity) or to proteins or nucleic acids which may function downstream of the BCL family member polypeptide, whether they are positively or negatively regulated by it. To illustrate, the critical residues of a subject BCL family member polypeptide which are involved in molecular recognition of interactor proteins upstream or downstream of a BCL family member (such as, for example BHI domains, BH2 domains) can be determined and used to generate BCL family member-derived peptidomimetics which competitively inhibit binding of the authentic BCL family member protein to that moiety. By employing, for example, scanning mutagenesis to map the amino acid residues of each of the subject BCL family member proteins which are involved in binding other extracellular proteins, peptidomimetic modulating agents can be generated which mimic those residues of the BCL family member protein which facilitate the interaction. Such mimetics may then be used to interfere with the normal function of a BCL family member protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted g lactam rings (Garvey et al. in Peptides: Chemistry and Biology, G. R. Marshall ed., ESCOM Publisher: Leiden, Netherlands, 1988). keto methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1: 1231), and b-aminoalcohols (Gordon et al. (1985) Biochem Biophys Res Commun 126:419; and Dann et al. (1986) Biochem Biophys Res Commun 134:71).

Other exemplary bcl modulating agents include any compounds which, when contacted with a cell, alter the “bioactivity” of a bcl family member protein. For example, the bioactivity of a bcl family member can be increased by turning on a bcl family member gene and increasing its transcription, stabilizing a bcl family member mRNA, increasing the rate of bcl family member protein synthesis, decreasing the rate of bcl family member protein degradation, animating bcl family member functions, helping proper folding of a bcl family member protein, aiding a bcl family member protein in reaching its subcellular compartment(s) promoting bcl family member interactions with relevant targets, such as for example Raf-I (Wang et al. 1996 Cell 87:629), and/or activating directly or indirectly targets downstream of a bcl family member.

The term “bioactivity” of a bcl family member is meant to include the ability of a molecule to promote axonal growth. Increases in the bioactivity of a bcl family member can occur absent any alteration in transcription of a bcl family member.

For example, bioactivity can be altered by allosteric molecules which bind to or interact with a bcl family member. Bioactivity of a bcl family member can also be assessed by its ability to compete with a bcl-2 molecule in its ability to promote axonal growth.

Competition with a bcl-2 molecule can be tested, for example in cells which express bcl 2 and a bcl family member and inhibition of axonal growth can be quantitated.

A preferred agent that increases bcl-2 expression that can be used according to the methods of the invention is lithium or analog or salt thereof. Exemplary salts of lithium that can be used include lithium chloride, lithium acetate, lithium carbonate, lithium citrate and lithium sulfate. For example, lithium chloride (LiCl) can be administered to a subject having a state characterized by diminished potential for axonal growth. Numerous salts of lithium are commercially available for treating certain manic-depressive illnesses. Compounds having structural similarities to lithium or a salt thereof can also be used according to the invention. Such alternative compounds can be tested according to methods described herein. They may also be tested for their ability to increase bcl-2 expression. Lithium or analogs or salts thereof can be administered sytemically or locally. For example, lithium can be administered at the site of a neural injury, e.g., a spinal cord injury. Thus, lithium can be administered with a syringe or a stent (e.g., coated stent) to the site of injury. Such compounds can also be administered at the site of the injury during reparative surgery. They can also be administered at the site where the bodies of the neural cells are from which the axons were severed. For example, two nerve endings can be brought within a certain distance from one another, e.g., within less than about 10 mm, preferably less than about 6 mm, 3 mm, 1 mm, 750 μm, 500 μm, 300 μm, 100 μm, 70 μm, 50 μm, 30 μm, 10 μm or less. Prior to, during or after bringing the nerve endings together, an amout of lithium, or salt or analog thereof, is added to the site where the nerve endings are brought together. The lithium can be present in a matrix for permitting slow release of the lithium.

The invention further provides compositions comprising lithium or a salt or analog thereof in a pharmaceutically acceptable excipient. The composition can further comprise an agent that creates an environment favorable to axonal growth, as further described herein.

Another agent that upregulates bcl-2 and can be used to promote axonal growth is valproate (valproic acid), which is commercially available (Manjii et al. (2001) Br J Psychiatry Suppl 2001 41:s107). Other agents for treating neurologic disorders could potentially also be used if these modulate the expression or activity of a bcl family member. Such agents include carbamazepine, lamotrigine, topiramate, gabapentin, primidone, benzodiazepine, clozapine, risperidone, calcium channel blockers, (such as verapamil, diltiazem, nifedipine, and nimodipine), bupropion, serotonin reuptake inhibitor, monoamine oxidase inhibitor, venlalfaxine, nefazodone, tricyclic antidepressants.

Still other bcl modulating agents are molecules which influence the bioactivity of a bcl family member protein indirectly, by modulating molecules which bind to a bcl family member in order to effect changes in the bioactivity of a bcl family member. Exemplary agents which bind to and alter the bioactivity of bcl family members include Bax, Bak, Mcl-1, Bag, Nip 1, Nip 2, and Nip 3 (Farrow and Brown Curr Opin in Genetics and Dev. 6:45 (1996)). For example, Raf-I has also been found to interact with bcl-2 (Gajewski and Thompson. 1996. Cell 87:589). Therefore, the present invention also provides for modulating bcl family members by modulating proteins which interact with and affect the bioactivity of a bcl family member, such as by changing the ratio between a bcl family member and proteins with which they interact.

In another embodiment, the invention provides methods for preventing neural cell degeneration essentially without stimulating regeneration. The method can comprise contacting the neural cell with a compound that increases expression or bioactivity of Bcl-x_{L}. For example, the method may comprise contacting a neural cell with a compound that increases the expression of Bcl-x_{L}. The method may also comprise introducing into the cell a nucleic acid encoding a Bcl-x_{L} protein or portion thereof sufficient for preventing neural cell degeneration. In other embodiments, the invention comprises introducing into a neural cell a Bcl-x_{L} protein or portion thereof.

In other embodiment, a neural cell is contacted with an agent that increases the expression and bioactivity of bcl2 and an agent that increases the expression and bioactivity of Bcl-x_{L}.

In yet another embodiment, this invention also teaches methods to screen for pharmacologically acceptable agents that can reach the CNS and turn on a bcl family member gene, stabilize bcl family member mRNA, increase rate of bcl family member protein synthesis, decrease bcl family member protein degradation, enhance bcl family member bioactivity, animate bcl family member functions, help proper folding of bcl family member protein, aid bcl family member protein to reach its subcellular compartment(s), promote bcl family member interactions with relevant targets, such as Raf-I at mitochondria (Wang et al. 1996 Cell 87:629), and/or activate directly or indirectly targets downstream of a bcl family member. Neurons cultured in Terasaki plates, 96-well plates, and recently developed 864-well plates may be used for screenings of a larger number of agents for any or all of biological activities listed above. Agents appropriate for such screenings include any of the 21-million structures listed in Chemical Abstract Database, any natural products, large or small, derived from animals, plants. microorganisms, marine organisms, insects, fermentation or biotransformation, or any fixture molecules to be generated by conventional organic synthesis, rational drug design or combinatorial chemistry. Robotic high-throughput and ultrahigh-throughput screening methods may be employed to identify such pharmacological agents with desirable activities that promote CNS regeneration via a bcl family member pathway.

Assay endpoints for robotic screenings include, but are not limited to, increased expression of a bcl family member (by immunofluoresence or immunoperoxidase with antibodies specific for bcl family member protein), increased mitochondrial membrane potentials (a consequence of increased bcl family member expression that can be detected by fluorescent, delocalized lipophilic cations), resistance to uncouplers for oxidative phosphorylation such as dinitrophenols or FCCP (a consequence of increased bcl family member expression that can be monitored by fluorescent dyes), resistance to apoptosis inducers (a consequence of increased bcl family member expression measurable by MTT or MTS dyes), and/or increased neural regeneration and neurite outgrowth.

Active compounds revealed by the assays listed above shall be further characterized by comparing their effects on neurons derived from uncompromised mice, bcl family member (−/−) knockout mice, or bcl family member transgenic mice. Pharmacological agents that promote neural regeneration via a bcl family member or its mRNA or its protein should be inactive in bcl-2 family member (−/−) knockout mice. Agents that turn on a bcl family member gene should be active in neurons derived from uncompromised mice. Agents that stabilize bcl family member mRNA or proteins should be active in neurons derived from bcl family member transgenic mice.

Pharmacological agents that animate bcl family member function or activate targets downstream of bcl family member may still be active in bcl family member knockout mice. Thus, this invention embodies any screening methods that allow the identification of any molecules, large or small, naturally occurring or man-made (by conventional organic synthesis or combinatorial chemistry), that act on bcl family member pathway in neurons, be it at bcl family member gene or its mRNA or its protein, or at bcl family member protein's downstream targets, and are able to induce their regeneration.

In other embodiments of the invention, members of the bcl family which can function to promote axonal growth can be identified in axonal growth screening assays (AGSAs), such as the co-culture system described in the examples. In the subject AGSAs, first a tissue sample, which contains the source of axons, is placed in contact with a second tissue sample into which said axons can grow. The expression of a bcl family member can be modulated in the first tissue sample and the effects thus can be selected on axonal growth can be determined. Thus, bcl family members can be selected which have a bcl bioactivity, e.g., promote axonal growth. Axonal growth can be measured by determining or quantifying the extension of axon(s), for example, as described in the appended Exemplification.

The subject AGSAs can also be used to select agents which can modulate axonal growth by providing a first tissue sample which contains axons and abutting it with a second tissue sample into which said axons can grow. Various agents can then be tested for effects on axonal growth by addition of the agents to the culture and agents which promote axonal growth can be selected. Such agents may be obtained, for example, through rational design or random drug-screening. The modulation of bcl family member bioactivity can occur either in vitro or in vivo.

In one embodiment a bcl family member can be modulated in a neural cell in vitro. Bcl modulation can be tested by measuring a bcl bioactivity in the cells (i.e., the promotion of axonal growth) or by performing immunoblot analysis, immunoprecipitation, or ELISA assays. The neural cell can be transplanted into a subject who has suffered a traumatic injury or with a state characterized by diminished axonal growth.

In certain embodiments, expression or activity of bcl2 is increased in a cell, e.g., a neural cell in vitro. For example, cells can be obtained from a subject, treated in vitro to increase bcl2 expression or activity, such as by introducing into the cells a nucleic acid encoding a bcl2 protein or by treating the cells with a lithium salt, so as to start axon elongation. The cells can then be administered back into the same or another subject. This may be helpful when neural cells are transplanted into a subject. In another embodiment, a stem cell, such as an embryonic stem (ES) cell or a germ cell is induced to differentiate into neural cells and the cells are induced to grow axons by increasing the level of bcl2 in the cells. ES can be differentiated into neural (or neuronal) cells according to methods known in the art. Differentiated neural cells having at least a portion of an axon can then be implanted into a subject in need thereof, e.g., a subject having a CNS injury.

The invention provides agents, compositions and methods for use in improving nerve regeneration or promoting nerve survival, in treating peripheral nerve injury and spinal cord injury, and in stimulation of growth of endogenous, implanted or transplanted neural tissue, e.g., CNS tissue. The present invention therefore also provides a method of promoting regeneration of an injured or severed nerve or nerve tissue, or promoting axon growth in neural (or neuronal) cells under a variety of neurological conditions requiring axon growth or prevention of neural cell degeneration.

As used herein, the term “state characterized by diminished potential for axonal growth” is meant to encompass a state or disorder which would benefit from the axonal growth induced by increased expression of a bcl family member. Reduced expression of a bcl family member may occur normally, as in adult neurons of the CNS, or because of a pathologic condition brought about by the misexpression of a bcl family member. “Diminished” as used herein is meant to include states in which axonal growth is absent as well those in which it is reduced. The present invention specifically provides for applications of the method of this invention in the treatment of states characterized by diminished potential for axonal growth. Exemplary states “characterized by diminished potential for axonal growth” include neurological conditions derived from injuries of the spinal cord or compression of the spinal cord, or complete or partial transection of the spinal cord. For example, injuries may be caused by: (i) acute, subacute, or chronic injury to the nervous system, including traumatic injury (e.g. severing or crushing of a neuron(s)), such as that brought about by an automobile accident, fall, or knife or bullet wound, (ii) chemical injury, (iii) vascular injury or blockage, (iii) infectious or inflammatory injury such as that caused by a condition known as transverse myelitis, or (iii) a tumor-induced injury, whether primary or metastatic. Thus, injuries leading to a state associated with diminished potential for axonal growth can be direct, e.g., due to concussion, laceration, or intramedullary hemorrhage, or indirect, e.g., due to extramedullary pressure of loss of blood supply and infarction.

The present invention will be useful in treating neurons in both the descending (e.g., corticospinal tract) and ascending tracts (e.g., the dorsal column-medial lemniscal system, the lateral spinothalarnic tract, and the spinocerebellar tract) of the spinal cord and in the reestablishment of appropriate spinal connections.

Common mechanisms of spinal cord injury include fractures of the vertebrae, which can damage the spinal cord from the concussive effect of injury due to displaced bony fragments, or damaged blood vessels, or contusion of emerging nerve roots. Dislocation of vertebrae can also cause spinal cord damage; dislocation is often the result of the rupture of an intervertebral disk, and may result in partial or complete severance of the spinal cord. Penetrating wounds can also cause severance. or partial severance of the cord. Epidural hemorrhage and spinal subdural hematoma can result in progressive paraparesis due to pressure on the spinal cord. Examples of indirect injury to the spinal cord include damage induced by a blow on the head or a fall on the feet.

Intramedullary injury can be the result of direct pressure on the cord or the passage of a pressure wave through the cord, laceration of the cord by bone, or the rupture of a blood vessel during the passage of a pressure wave through the cord with a hemorrhage into the cord. Intramedullary bleeding and hematoma formation can also be caused by rupture of a weakened blood vessel. Ischemic damage can occur following compression of the anterior spinal artery, pressure on the anastornotic arteries, or damage to major vessels (Gilroy, in Basic Neurolog5, McGraw-Hill, Inc. New York, N.Y. (1990).

The present invention will also be useful in promoting the recovery of subjects with a herniated disks, hyperextension-flexion injuries to the cervical spine and cervical cord, and cervical spondylosis. In addition to treating movement disorders, the present invention will be use” in treating disorders of the brain, e.g. the brain stem and in enhancing brain or brain stem function in a subject with a state characterized by diminished potential for axonal growth. For example, the present invention can be used in the treatment of brain damage. For example, the brain damage can be caused by stroke, bleeding trauma, or can be tumor-related brain damage. The present invention will also be useful in treating peripheral neuropathies. Damage to peripheral nerves can be temporary or permanent and, accordingly, the present invention can hasten recovery or ameliorate symptoms.

Peripheral neuropathies include, among others, those caused by trauma, diabetes mellitus, infarction of peripheral nerves, herniated disks, epidural masses, and postinfectious (or postvaccinal) polyneurites. The symptoms of peripheral neuropathies which will benefit from the instant invention include muscle wasting and weakness, atrophy, the appearance of fasciculations, impaired tendon reflexes, impaired sensation, dysethesias or paresthesias, loss of sweating, alteration in bladder function, constipation, causalgia, and male impotence.

In another embodiment, the methods and compositions are used for treating glaucoma, a neuropathy that causes blindness. Glaucoma is characterized by the excavation of the optic disk and degeneration of retinal ganglion cells. High intraocular pressure is considered to be a risk factor for developing this disease. In one embodiment, a subject having glaucoma or susceptible to developing glaucoma is treated by the administration of a composition of the invention, e.g., a bcl-2 gene, protein or homolog or fragment thereof or lithium, a derivative or analog thereof or salt thereof (the “therapeutic compound”). The therapeutic compound can be administered to a subject via various modes. For example, at least certain compounds, such as LiCl, may be administered orally to the patient. At least certain therapeutic compounds can be applied directly to the eye(s) of the subject. Other routes of administration are further described herein. In another embodiment, a therapeutic compound is administered by injection into the vitreous chamber of the eye(s) of the subject. The amount of therapeutic compound to be administered can be based on studies in animal models of glaucoma (as further described herein) as well as in medical trials.

The use of the instant invention to treat neurodegenerative diseases which will benefit by enhanced axonal growth is also provided for. In preferred embodiments the subject invention is used to treat neurodegenerative diseases, such as, Pick's disease, progressive aphasia without dementia, supranuclear palsy, Shy-Drager Syndrome, Friedreich's ataxis, olivopontocerebellar degeneration, vitamin E deficiency and spinoecrebellar degeneration, Roussy-Levy Syndrome, and hereditary Spastic ataxia or paraparesis. In addition, treatment of other disorders of the spinal cord, such as amyotrophic lateral sclerosis, spinal muscular atrophies, and multiple sclerosis are intended to be part of the present invention. In other embodiments the present invention will be useful in ameliorating the symptoms of neural degeneration such as that induced by vitamin B 12 deficiency, or associated with HIV infection (AIDS), or HTLV-I infection. In particularly preferred embodiments of the present invention are used to treat any neurodegenerative disorder with the exception of Alzheimer's disease, Parkinson's disease, cancer, or viral infections. The anti-apoptotic treatment of Alzheimer's disease, Parkinson's disease, cancer, or viral infection are intended to be part of this invention.

Other states characterized by diminished potential for axonal growth which will benefit by the present invention will be apparent to one of ordinary skill in the art. The term “treatment” is intended to include prevention and/or reduction in the severity of at least one symptom associated with the state being treated. The term also is intended to include enhancement of the 10 subject's recovery from the state.

The term “subject” as used herein is meant to encompass mammals. As such the invention is useful for the treatment of domesticated animals, livestock, zoo animals, etc. Examples of subjects include humans, cows, cats, dogs, goats, and mice. In preferred embodiments the present invention is used to treat human subjects. Subjects can be adults, children, neonates, or fetuses. In certain embodiments, the subject is a human subject that is a neonate or child up to 1 day; 10 days; 1 month; 2 months; 3 months; 4 months; 5 months; 6 months; 9 months 1, 2 or 3 years old. In certain embodiments, the injury is at a site in a subject in which the nerves are essentially not yet myelinated.

The present invention provides for the additional administration of agents which create an “environment” favorable to axonal growth. Exemplary agents include trophic factors, receptors, extracellular matrix proteins, intrinsic factors, or adhesion molecules. Exemplary trophic factors include NGF, BDNF, NT-3, 4, 5, or 6, CNTF, LIF, IGFI, IGFII, GDNF, GPA, bFGF, TGFB, and apolipoprotein E. Exemplary receptors include the Trk family of receptors. An exemplary extracellular matrix protein is laminin. Exemplary intrinsic factors include GAP-43 (also known as B 50, pp46, neuromodulin, and F I) and arneloid precursor protein (APP) (Moya et al. Del,. Biol. 161:597 (1994)). Exemplary adhesion molecules include NCAM and L 1. Nucleic acids encoding these polypeptides, or the polypeptides may be used. The use of peptide fragments of any of the above axonal growth enhancers could also be used.

In another embodiment the invention provides a method of treating a subject that has suffered a traumatic injury in which nerve cell injury has occurred, in which a subject is treated with a bcl modulating agent, e.g., such that axonal growth occurs. Exemplary traumatic injuries include severing or crushing of a neuron(s), such as that brought about by an automobile accident, fall, or knife or bullet wound, as well as others described herein. The present invention also provides a method of treating a subject for a state characterized by diminished potential for axonal growth by administering a therapeutically effective amount of an agent which modulates the bioactivity or expression of a bcl family member in a subject.

In one embodiment, the method of the invention comprises bringing within a certain distance the two ends of a severed nerve in a subject; and administering to the subject a nucleic acid, protein or compound of the invention, e.g., lithium (“therapeutic of the invention”). The distance is preferably a distance that regenerating axons can reach in a reasonable time. For example, the distance can be less than about 10 mm, preferably less than about 6 mm, 3 mm, 1 mm, 750 μm, 500 μm, 300 μm, 100 μm, 70 μm, 50 μm, 30 μm, 10 μm or less. Prior to, during or after bringing the nerve endings together, an amout of therapeutic of the invention is added to the site where the nerve endings are brought together. Nerve endings can be brought together by surgerical, e.g., microsurgical techniques.

In one embodiment, a severed or damaged nerve may be repaired or regenerated by surgically entubating the nerve in an entubalation device in which an effective amount of an agent of this invention can be applied to the nerve. In a related embodiment, an agent of the invention can be impregnated into an implantable delivery device such as a cellulose bridge, suture, sling prosthesis or related delivery apparatus. Such a device can optionally be covered with glia, as described by Silver, et al, Science 220:1067-1069, (1983). Bioabsorbable materials or matrices may be used in conjunction with the agents of the present invention to coat the interior of tubes used to connect severed neurons; they may be added directly to suture materials or incorporated in bioabsorbable materials in and on sutures; further, they may be utilized on/in implants and prosthetic devices, either alone or in conjunction with other bioabsorbable and supporting materials.

The composition containing the agent may be incorporated or impregnated into a bioabsorbable matrix, with the matrix being administered in the form of a suspension of matrix, a gel or a solid support. In addition, the matrix may be comprised of a biopolymer. In constructing the matrix, it may be useful for the matrix to further include a substructure for purposes of administration and/or stability. Suitable substructures include freeze dried sponge, powders, films, flaked or broken films, aggregates, microspheres, fibers, fiber bundles, or a combination thereof. In addition, the matrix may be attached to a solid support for administration purposes. Suitable supports depend upon the specific use and can include a prosthetic device, a porous tissue culture insert, an implant, a suture, and the like.

A therapeutically effective amount of a composition or agent of the invention is a predetermined amount calculated to achieve the desired effect, i.e., to effectively promote axon regeneration or preventing degeneration of targeted neuronal cells. In addition, an effective amount can be measured by improvements in one or more symptoms occurring in a patient.

The invention further contemplates an axon growth-promoting apparatus that comprises a bioabsorbable matrix and an effective amount of a pharmacologically active agent capable of inducing axon growth or preventing degeneration. The matrix can be in the form of a solid support and the pharmacologically active agent can be attached to the substrate. The agent can optionally be incorporated into the bioabsorbable matrix, which can be comprised of a biopolymer of a variety of materials. The matrix can further include a substructure comprising freeze dried sponge, powders, films, flaked or broken films, aggregates, microspheres, fibers, fiber bundles, or a combination, thereof. The solid support can be formulated into a prosthetic device, a porous tissue culture insert, an implant, an entubation apparatus and a suture. The matrix can be adapted for use in tissue culture.

Solid supports (also described as solid surfaces or solid substrates) useful according to the present invention include supports made of glass, plastic, nitrocellulose, cross-linked dextrans (e.g., SEPHADEX; Pharmacia, Piscataway, N.J.), agarose in its derivatized and/or cross-linked form, polyvinyl chloride, polystyrene, cross-linked polyacrylamide, nitrocellulose- or nylon-based webs such as sheets, strips or paddles, tubes, plates, the wells of a microtiter plate such as those made from polystyrene or polyvinylchloride, and the like, and may take the form of a planar surface or microspheres to name a few variations. Useful solid support materials in this regard include the derivatized cross-linked dextran available under the trademark SEPHADEX from Pharmacia Fine Chemicals (Piscataway, N.J.), agarose in its derivatized and/or cross-linked form, polystyrene beads about 1 micron to about 5 millimeters in diameter (available from Abbott Laboratories of North Chicago, Ill.), polyvinyl chloride, polystyrene, cross-linked polyacrylamide, nitrocellulose- or nylon-based webs such as sheets, strips or paddles, tubes, plates, the wells of a microtiter plate such as those made from polystyrene or polyvinylchloride, and the like.

In another embodiment, the invention discloses a method of preparing substrates (solid support) useful for promoting axon growth or preventing degeneration, comprising providing a composition containing an agent of this invention and treating by coating or impregnating a matrix in or on the solid substrate with said agent-containing composition. In various disclosed embodiments, the solid support or substrate may comprise glass, agarose, a synthetic resin material (e.g., nitrocellulose, polyester, polyethylene, and the like), long-chain polysaccharides, and other similar substances. The solid support can be formulated, as described herein, in a variety of administration formats for both in vitro or in vivo use, and the specific format need not be considered as limiting to the invention.

This invention also provides means for delivery of a bcl modulating agents to a neural cell. In certain embodiments gene constructs containing nucleic acid encoding a bcl family member are provided. As used herein the term “gene construct” is meant to refer to a nucleic acid encoding a bcl family member which is capable of being heterologously expressed in a neural cell. In certain embodiments, the a bcl family member may be operably linked to at least one transcriptional regulatory sequence for the treatment of a state characterized by diminished potential for axonal growth.

Operably linked is intended to mean that the nucleotide sequence is linked to a regulatory sequence in a manner which allows expression of the nucleotide sequence.

Regulatory sequences are art-recognized and are selected to direct expression of the subject bcl proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences-sequences that control the expression of a DNA sequence when operatively linked to it may be used in these vectors to express DNA sequences encoding the bcl polypeptides of this invention. Such useful expression control sequences, include, for example, a viral LTR, such as the LTR of the Moloney murine leukemia virus, the early and late promoters of SV40, adenovirus or cytornegalovirus PCT/US97/11814 immediate early promoter, the lac system, the trp system, the TAC or TRC system, T7 promoter whose expression is directed by T7 RNA polymerase, the major operator and promoter regions of phage I, the control regions for fd coat protein, the promoter for 3 phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. In preferred embodiments the promoter is designed specifically for expression in neural cells. In particularly preferred embodiments the promoter is a neural specific enolase promoter. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as markers, should also be considered.

In certain embodiments it will be desirable to additionally administer agents which create an environment favorable to axonal growth into an expression vector comprising a nucleic acid encoding a bcl family member. Examples of classes of such agents include trophic factors, receptors, extracellular matrix proteins, or intrinsic factors. Exemplary trophic factors include NGF, BDNF, NT-3, 4, 5, or 6, CNTF, LIF, IGFI, IGFII, GDNF, GPA, bFGF, TGFb, and apolipoprotein E. Exemplary receptors include the Trk family of receptors. An exemplary extracellular matrix protein is laminin. Exemplary intrinsic factors include GAP-43 and ameloid precursor protein (APP)(Moya et al. Dev. Biol. 161:597 (1994)). Exemplary adhesion molecules include NCAM and L1.

Agents which provide an environment favorable to axonal growth can be administered by a variety of means. In certain embodiments they can be incorporated into the gene construct. In other embodiments, they may be injected, either locally or systemically. In other embodiments such agents can be supplied in conjunction with nerve guidance channels as described in U.S. Pat. Nos. 5,092,871 and 4,955,892.

Accordingly, a severed axonal process can be directed toward the nerve ending from which it was severed by a prosthesis nerve guide which contains a non bcl agent as, e.g. a semi-solid formulation, or which is derivatized along the inner walls of the nerve guidance channel. These agents may be administered simultaneously with a bcl modulating agent, such as lithium, or not.

In certain embodiments of the invention, for example in the treatment of long-standing injury (e.g., when there has been significant colateral sprouting of a neural cell) it may be desirable to combine treatment with the subject bcl modulating agents with a “pruning procedure” to remove rostral sprouting (Schneider, G. E. Brain. Bahav Evol. 8:73 (1973)).

Expression constructs of the subject bcl modulating agents, may be administered in a biologically effective carrier, e.g. any formulation or composition capable of effectively delivering the bcl gene to cells in vivo. Approaches include insertion of the subject gene in viral vectors including recombinant retroviruses, adenovirus, adeno-associated virus, and herpes simplex virus-1, or other attenuated viruses, or recombinant bacterial or eukaryotic plasmids which can be taken up by the damaged axon. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO4 precipitation carried out in vivo. It will be appreciated that the choice of the particular gene delivery system will depend on such factors as the intended target and the route of administration, e.g. locally or systemically. In particularly preferred embodiments, the constructs employed are specially formulated to cross the blood brain barrier. Furthermore, it will be recognized that the gene constructs provided for in vivo modulation of bcl expression are also useful for in vitro modulation of bcl expression in cells, such as for use in the ex vivo assay systems described herein.

A preferred approach for in vivo introduction of nucleic acid into a cell is by use of a viral vector containing nucleic acid, e.g. a DNA, encoding the particular form of the bcl polypeptide desired. Infection of cells with a viral vector has the advantage that a large proportion of the targeted cells can receive the nucleic acid. Additionally, molecules encoded within the viral vector, e.g., by a DNA contained in the viral vector, are expressed efficiently in cells which have taken up viral vector nucleic acid.

Retrovirus vectors and adeno-associated virus vectors can be used as the gene delivery system of the present invention for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. Blood 76:271(1990). Thus, recombinant retrovirus can be constructed in which part of the retroviral coding sequence (gag, pol, env) has been replaced by nucleic acid encoding one of the subject receptors rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biolay, Ausubel, F. M. et al. (eds.) Greene Publishing Associates, (1989), Sections 9.10-9.14 and other standard laboratory manuals. Examples of retroviruses include pLJ, pZIP, pWE and pEM which are well known to those skilled in the art. Examples of packaging virus 1Q lines for preparing both ecotropic and amphotropic retroviral systems include yCrip, yCre, y2 and yAm. Retroviruses have been used to introduce a variety of genes into many different cell types in vitro and/or in vivo (see for example Eglitis, et al. Science 230:1395-1398(1985); Danos and Mulligan Proc. Natl. Acad. Sci. USA 85:6460-6464(1988); Wilson et al. Proc. Natl. Acad. Sci. USA 85:3014-3018(1988); Armentano et al. Proc. Natl. Acad Sci. USA 87:6141 6145(1990); Huber et al. Proc. Natl. Acad. Sci. USA 88:8039-8043(1991); Ferry et al. Proc. Natl. Acad. Sci. USA 88:8377-8381(1991); Chowdhury et al. Science 254:1802 1805(1991); van Beusechem et al. Proc. Natl. Acad Sci. USA 89:7640 7644(1992); Kay et al. Human Gene Therapy 3:641-647(1992); Dai et al. Proc. Natl. Acad Sci. USA 89:10892-10895(1992); Hwu et al. J. Immunol. 150:4104-4115(1993); U.S. Pat. No. 4,868,116; U.S. Pat. No. 4,980,286; PCT Application WO 89/07136; PCT Application WO 89/02468, PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. PNAS 86:9079 9083(1989); Julan et al. J. Gen Virol 73:3251-3255(1992); and Goud et al. Virology 163:251-254(1983)); or coupling cell surface receptor ligands to the viral env proteins (Neda et al IBiol Chem 266:14143-14146(1991)). Coupling can be in the form of the chemical cross-linking with a protein or other variety (e.g. lactose to convert the env protein to an asialoglycoprotein), as well as by generating fusion proteins (e.g. single in chain antibody/env fusion proteins). This technique, while useful to limit or otherwise direct the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the bcl gene of the retroviral vector.

Another viral gene delivery system useful in the present invention utilitizes adenovirus-derived vectors. The genome of an adenovirus can be manipulated such that it encodes and expresses a gene product of interest but is inactivated in terms of its ability to replicate in a normal lytic viral life cycle. See for example Berkner et al. Bioltechniques 6:616(1988); Rosenfeld et al. Science 252:431 434(1991); and Rosenfeld et al. Cell 68:143-155(1992). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types (Rosenfeld et al. supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham J Virol. 57:267(1986)). Most replication defective adenoviral vectors currently in use and therefore favored by the present invention are deleted for all or parts of the viral E I and E3 genes but retain as much as 80% of the adenoviral genetic material (see, e.g., Jones et al. Cell 16:683(1979); Berkner et al., supra; and Graham et al. in Methods in Molecular Biology, E. J. Murray, Ed. (Humana, Clifton, N.J., 1991) vol. 7. pp. 109-127). Expression of the inserted bcl gene can be under control of, for example, the E I A promoter, the major late promoter (MLP) and associated leader sequences, the E3 promoter, or exogenously added promoter sequences.

Yet another viral vector system useful for delivery of the subject bcl gene is the adeno-associated virus (AAV). Adeno-associated virus is a naturally occurring, defective virus that requires another virus, such as an adenovirus or a herpes virus, as a helper virus for efficient replication and a productive life cycle. (For a review see Muzyczka et al. Curr. Topics in Micro. and Immunol. 158:97-129(1992)). It is also one of the few viruses that may integrate its DNA into non-dividing cells, and exhibits a high frequency of stable integration (see for example Flotte et al. Am. 0.1 Respir. Cell. Mol. Biol. 7:349-356(1992); Samulski et al. J. Virol. 63:3822-3828(1989); and McLaughlin et al. J. Virol. 62:1963-1973 (1989)). Vectors containing as little as 300 base pairs of AAV can be packaged and can integrate. Space for exogenous DNA is limited to about 4.5 kb. An AAV vector such as that described in Tratschin et al. Mol. Cell. Biol. :3251-3260 (1985) can be used to introduce DNA into cells. A variety of nucleic acids have been introduced into different cell types using AAV vectors (see for example Hermonat et al. Proc. Natl. Acad Sci. U. A. 81:6466-6470(1984); Tratschin et al. Mol. Cell. Biol. 4:2072-2081(1985); Wondisford et al. Mol. Endocrinol. 2:32 39(1988); Tratschin et al. J Virol. 51:611-619 (1984); and Flotte et al. J. Biol. Chem. 268:3781 3790(1993)).

Replication defective Herpes simplex virus-1 (HSV-1) vectors have been shown to achieve efficient transduction and expression of heterologous genes in the nervous system (Dobson et al. Neuron. 5:353(1990); Federoff et al. Proc. Nat Acad Sci. U.S.A. 89:1636(1992); Andersen et al. Hum Gene Ther. 3:487(1992); Huang et al. Exl) Neurol. 115:303(1992); Fink et al. Hum Gene Ther. 3:11(1992); Breakefield et al. in Gene Transfer and Therapy in the Nervous System. Heidelberg, FRG: Springer Verlagpp 45-48(1992); and Ho et al. Proc Naff Acad Sci U.S.A. 90:3655(1993)). HSV-2 vectors expressing bcl have also been described (Linnik et al. Stroke. 26:1670(1995); Lawrence et al. J Neuroscience. 16:486(1996)).

In addition to viral transfer methods, such as those illustrated above, non viral methods can also be employed to cause expression of a bcl polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject bel polypeptide gene by the targeted cell. Exemplary gene delivery systems of this type include liposomal derived systems, poly-lysine conjugates, and artificial viral envelopes.

In a representative embodiment, a gene encoding the subject bcl polypeptides can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 10473 81; and European patent publication EP-A-43075). For example, lipofection of cells can be carried out using liposomes tagged with monoclonal antibodies against any cell surface antigen present on the target cells.

In one aspect, the invention features a pharmaceutical preparation which includes a recombinant transfection system. The term “recombinant transfection system” is meant to include a gene construct including a nucleic acid encoding a bcl modulating agent, a gene delivery composition, and, optionally one or more non-bc/agents as described herein, which create an environment favorable to axonal growth. Such “gene delivery compositions” are capable of delivering a nucleic acid encoding a bcl family member to its intended target, e.g., a neural cell and can include the compositions described herein, such as, a viral vector or recombinant bacterial or eukaryotic plasmids. Plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO4 precipitation.

In clinical settings, the gene delivery systems for the therapeutic bcl gene can be introduced into a subject by a number of methods, each of which is art recognized. For instance, a pharmaceutical preparation of the gene delivery system can be introduced systemically, e.g. by intravenous injection, and specific transduction of the nucleic acid in the target cells occurs predominantly from specificity of transtection provided by the gene delivery composition, site of administration, cell type or tissue type expression due to the transcriptional regulatory sequences controlling expression of the receptor gene, or a combination thereof. In other embodiments, initial delivery of the recombinant gene is more limited with introduction into the animal being quite localized, for example delivery can be targeted to a specific area of the brain, e.g., the injection can be intraventricular. To facilitate local delivery, the gene delivery vehicle can be introduced by stereotactic injection (e.g. Chen et al. PNAS 91: 3054-3057(1994)).

The pharmaceutical preparation of the gene delivery composition can contain the gene delivery system in an acceptable diluent, or can contain a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

Pharmaceutical compositions containing a bcl family member polypeptide and a pharmaceutically acceptable carrier formulated for promoting axonal growth also are intended to be part of this invention. The compositions can contain the full length protein or the fragments described above. The pharmaceutical compositions containing the polypeptide can be formulated to target a neural cell, or can be specially formulated for an anti-apoptosis use such as those described herein. For example, the peptide can be conjugated for example, to a carrier or encapsulated within a delivery system.

Pharmaceutical compositions for use in accordance with the present invention may be formulated in a conventional manner using one or more physiologically acceptable carriers or excipients. Thus, the compounds and their physiologically acceptable salts and solvates may be formulated for administration, for example, by injection.

For example, the compositions of the invention can be formulated for a variety of loads of administration, including systemic. Techniques and formulations generally may be found in Remminglons Pharmaceutical Sciences, Meade Publishing Co., Easton, Pa. For systemic administration, injection is preferred, including intramuscular, intravenous, intraperitoneal, and subcutaneous. For injection, the compositions of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

The compositions may be formulated for parenteral administration by injection, e.g., by bolus injection or continuous infusion. Formulations for injection may be presented in unit dosage form, e.g., in ampules or in multi-dose containers, with an added preservative. The compositions may take such forins as suspensions, solutions or emulsions in oily or aqueous vehicles, and may contain formulation agents such as suspending, stabilizing and/or dispersing agents. Alternatively, the active ingredient may be in powder form for constitution with a suitable vehicle, e.g., sterile pyrogen-free water, or saline before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosages for use in humans. For example, the dosage of such compositions lies preferably within a range that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma or local tissue concentration range that includes the IC50 (i.e., the concentration of the test compound which achieves a half-maximal therapeutic effect, e.g., inhibition of symptoms) as determined in cell culture. Such information can be used to more accurately determine useful doses in humans. Levels in plasma or local tissue may be measured, for example, by high performance liquid chromatography. The regimen of administration can also affect what constitutes an effective amount. The compositions of the present invention can be administered in several divided dosages, as well as staggered dosages, can be administered daily or sequentially, or the dose can be continuously infused, or can be a bolus injection. Further, the dosages of the agent(s) can be proportionally increased or decreased as indicated by the exigencies of the therapeutic or prophylactic situation. Another embodiment of the present invention provides for a packaged drug for the treatment of a state associated with diminished potential for axonal growth, which includes a bcl modulating agent packaged with instructions for treating a subject.

The “packaged drug” of the present invention can include any of the compositions described herein. The term “instructions” as used herein is meant to include the indication that the packaged drug is useful for treating a state associated with diminished potential for axonal growth and optionally may include the steps which one of ordinary skill in the art would perform to treat a subject with such a state.

The present invention is further illustrated by the following examples, which should not be construed as limiting in any way. The contents of all cited references including literature references, issued patents, published and non published patent applications as cited throughout this application are hereby expressly incorporated by reference. The animal models used throughout the Examples are accepted animal models and the demonstration of efficacy in these animal models is predictive of efficacy in humans.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### EXAMPLES

### Example 1

### Growth of Retinal Axons

To examine the growth of CNS axons of mice, an organotypic coculture model of the retinotectal system was established, in which the growth pattern of retinal axons closely mimics that seen in vivo (Chen, D. F., Jhaveri, S. & Schneider, G. E. Proc. Natl Acad Sci. USA 92, 7287-7291 (1995)). Tissues from retinae and midbrain tecta of C57BL/6J mice are abutted in a culture well (see Example 6 for details on the methods). Quantitative analysis of axonal growth from retinae is achieved by the standard placement of Dil into retinal explants. Cocultures prepared from animals aged embryonic day 14 (E 14, day of mating=EO) through E16 were examined. Growth of retinal axons into the tectal slice was extensive (n=20); axons for E16 retinae could be observed growing into the entire tectal explant, and the number of labeled axons invading tectal tissue averaged 126+/−10.0. In contrast, retinal explants (n=60) prepared from animals at age E 18 and older exhibited markedly reduced axonal growth. For E 18 tissues, the mean result was averaging 15.5+/−3.3 fibers per tectal slice, while no obvious increase in cell death was observed in these cultures. This indicated that starting at E 18 in mice, most RGC axons display a regenerative failure in culture. Thus, the level of expression of bcl-2 in RGCs correlates with the growth ability of retinal axons. This finding matched the previous report on the Syrian hamster (Chen et al. supra). Previous work showed that embryonic RGCs can grow axons into tectal tissue of any age, whereas older retinae fail to grow many axons into CNS tissue of any age including into embryonic tecta. To determine which genes might play such roles in regulating the growth of retinal axons, the level of expression of several molecules, including bcl-2, was compared with the use of immunofluorescence staining. High expression of bcl-2 at E 16 in the RGC layer of retinae was found. At E 18, in parallel with the onset of regenerative failure in culture, the expression of bcl 2 decreased to an undetectable level.

The retino-brain slice co-culture system described herein circumvents problems encountered with classic primary cell cultures by using retinal explants that maintain inter-cellular interactions and provide regenerating axons a nature environment (brain slice) for navigation. Retino-brain slice co-cultures offer an advantageous culture system that resembles the in vivo regenerative process of severed optic nerves and facilitates the drug screening process.

### Example 2

### A bcl Family Member is Required for the Growth of Axons

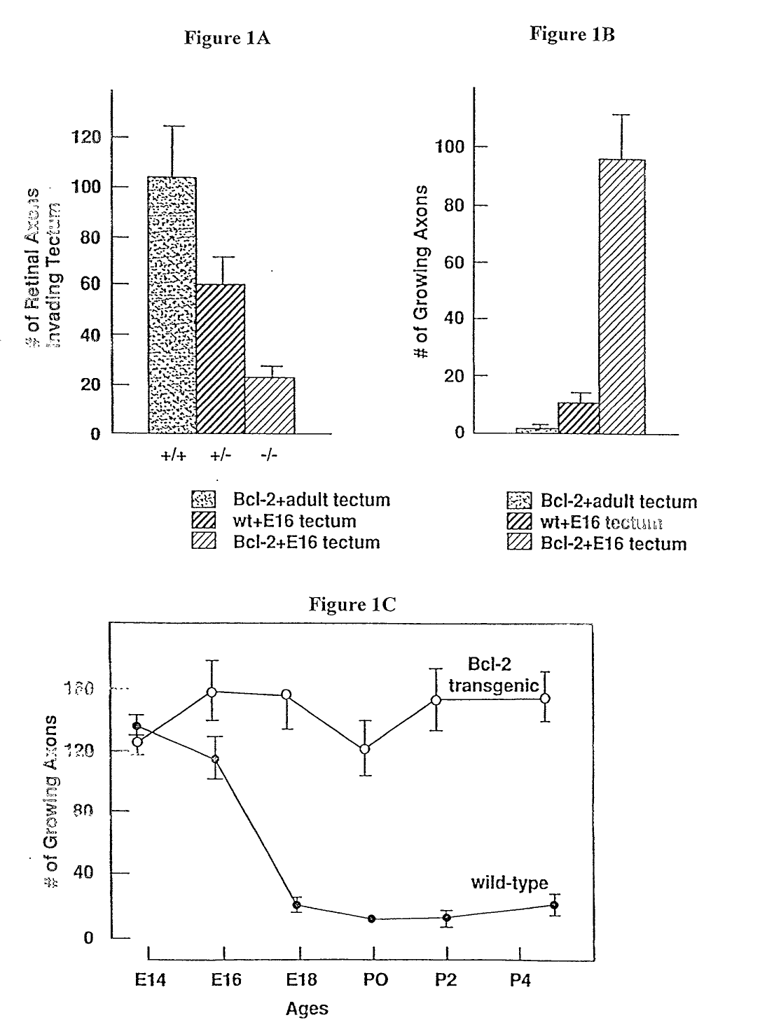

To determine whether bcl-2 is required for the growth of retinal axons, loss-of function animal model—mice genetically deficient in bcl-2 was studied (Veist et al. Cell 75, 229 240 (1993)). These mice were derived from matings of heterozygous offspring. Resulting litters contained wild-type, heterozygous, and bcl-2-deficient mice. Cocultures were prepared from E 15 embryos. At this stage, retinal explants of wild-type animals showed robust neurite outgrowth. To exclude the possibility that tectal tissues from mutant mice may affect axonal growth of RGCs, a series of parallel experiments was performed in which retinal explants from each animal had the possibility of being cocultured with the tectum from a wild-type, heterozygous, or homozygous animal. Regardless of the origin of tectal tissue, retinal explants derived from embryos of heterozygous and homozygous bcl-2 mutation grew significantly fewer neurites than those from wild type littermates (P<0.001). The numbers of labeled retinal axons were reduced by 50% in retinae prepared from heterozygous animals (62+−8, n=20) and by 80% in those from homozygous animals (22+/−4, n=7) (FIG. 1A). There was no significant difference between groups of retinae cocultured with tecta from wild-type and mutant mice. It should be noted that the numbers of retinal axons from cultures of mice containing the homozygous bcl-2 mutation were equivalent to those of wild-type mice on E 18—when most RGCs failed to grow axons into tectum.

### Example 3

### Expression of a bcl Family Member Allowed Axon Regeneration in Adult Neural Tissue

Since loss of bcl-2 function represses axonal growth, whether or not overexpression of bcl-2 in adult retinae is sufficient for retention of retinal axon regeneration was tested. Therefore, mice transgenic for the bcl-2 gene driven by the neuron-specific enolase promoter (Martinou, J-C. et al. Neuron 13, 1017 1030 (1994); Dubois-Dauphinet al. Proc. Natl. Acad. Sci. USA 91, 3309-3313 (1994)) were analyzed. The study was performed on line 73 of these transgenic mice. A series of timed matings was set up between males heterozygous for the transgene and wild-type (C57BL/6J) females. Half of the pups derived from these matings were transgenic. Cocultures of retinae and tecta derived from animals aged E 14 through postnatal day 5 (P5, day of birth=PO), which covered the period before and after regenerative failure normally occurs were examined. As previously described, the experiment was designed so that retinal explants from each mouse had the possibility of being cocultured with tecta of wild-type or transgenic mice. Starting at E 18, retinal explants from wild-type mice exhibited a failure of RGC axon elongation (n=15), regardless of whether confronted with wild-type or transgenic tectal tissues (FIG. 1C). The number of labeled retinal axons decreased 10 fold in comparison to E16 retinal explants. In contrast, when retinae were derived from bcl-2 transgenic animals, all retinal explants, harvested from animals aged E14 through P5, showed extensive fiber outgrowth (n=35) (FIG. 1C). No difference was observed in the numbers of retinal axons that invaded tectal slices derived from wild-type and bcl-2 transgenic mice. Therefore, constitutive expression of bcl-2 in RGCs, rather than in the CNS environment of the axon, overcomes regenerative failure of retinal axons in the perinatal period. RGCs derived from bcl-2 transgenic mice retained the ability to grow axons throughout their life span. Extensive neurite outgrowth was observed from adult retinal explants of transgenic mice when they were cocultured with E 16 tectal slices (n=10); the number of labeled retinal axons averaged 96.3+/−15.3, almost equivalent to the number obtained from an E16 retinotectal coculture. However, when the adult retinae were confronted with adult tectal tissues, little axonal growth was achieved (n=13) (FIG. 1B). This indicates that retinal axons of bcl-2 overexpressing mice have the ability to grow only into tissues expressing very permissive substrates, as presumably provided by the embryonic tectum. Therefore, bcl-2 is not the sole protein responsible for the regeneration of CNS axons in adult; it is probable that adult CNS contains inhibitory signals suppressing the regrowth of retinal axons from transgenic mice (Schnell, L. & Schwab, M. E. Nature 343, 269-272 (1990)). Thus, bcl-2 plays a central role in regulating the intrinsic genetic program for retinal axonal growth. Bcl-2 is essential but not sufficient for the regeneration of retinal axons in mature CNS under the conditions tested in this example (for this particular neural cell type and this particular bcl family member).

In this model of retino-brain slice (tectal) co-cultures, in which a retinal explant is placed directly against a brain slice containing the target area of RGC axons, the SC (tectum), virtually, all of the connections between the retina and the brain are disrupted prior to culture preparation. Thus, any axons that grow from a retinal explant into the brain slice after culturing reflect regenerative activity of retinal axons. This co-culture model provides regenerating axons a nature environment, the brain slice, for navigation and undisrupted cell-cell interactions within the retina. Axonal growth from the retina into the brain slices in this model largely resembles the process of optic nerve regeneration in vivo. In addition, the co-culture system offers many advantages, even over that of the in vivo model, including easy assessment, manipulation, and quantitation for axonal regeneration and consistent growing conditions.

### Example 4

### A bcl Family Member Promoted Axonal Growth in vivo

Subsequently, the regeneration of retinal axons in vivo was studied. Young pups (P4) obtained from the mating of males heterozygous for the bcl-2 transgene and C57BL/6J females, received a unilateral transection of the optic tract at the mid-tectal level. Axonal regrowth was assessed by tracing of retinal projection fibers with cholera toxin B-subunit (CT-13) (Angelucci et al. J Neurosci. Meth. 65, 101-112 (1996)). To visualize the lesion site, every other sagittal section of these brains was collected for cresyl violet staining and reconstructed in three dimensions with the Neurotrace program. In wild-type mice, the retinotectal projection was visible but was restricted to the tissue proximal to the lesion site (n=5). In contrast, axotomized retinal axons in transgenic mice grew in large numbers across the lesion site and innervated the tectum caudal to the injury (n=6). Thus, expression of bcl-2 in transgenic mice led to regeneration of retinal axons after optic tract transection in vivo. While in wild-type animals labeled axons did not cross the lesion site, those from bcl transgenic mice regenerated across the lesion site and entered the caudal tecturn. In three transgenic mice, the lesion produced a large, impassable gap in the superficial superior colliculus, but nevertheless the axons were observed to curve around the lesion site en route to the target tissue, without the addition of any bridging material or neurotrophic factors. Many axons reached the posterior border of the superior colliculus (SC). No axons were observed to invade the inferior colliculus. These results demonstrated that bcl-2 promoted retinal axon regeneration in vivo. It should be emphasized that in the above examples, large numbers of RGCs in wild-type animals survived after injury, but seemed unable to regenerate their axons. Similar observations have been reported by other investigators (Misantone et al. J. Neurocytol. 13, 449-465 (1984); Winkler et al. Dev. Brain Res. 28,11-21 (1986); Harvey, A. R. & Robertson, D. J Comp. Neurol. 325, 83-94 (1992)), who suggested a dissociation of neuronal survival and axonal regrowth after axotomy.

### Example 5

### Effects of a bcl Family Member on Neuron Survival and Axonal Growth can be Distinguished in vitro

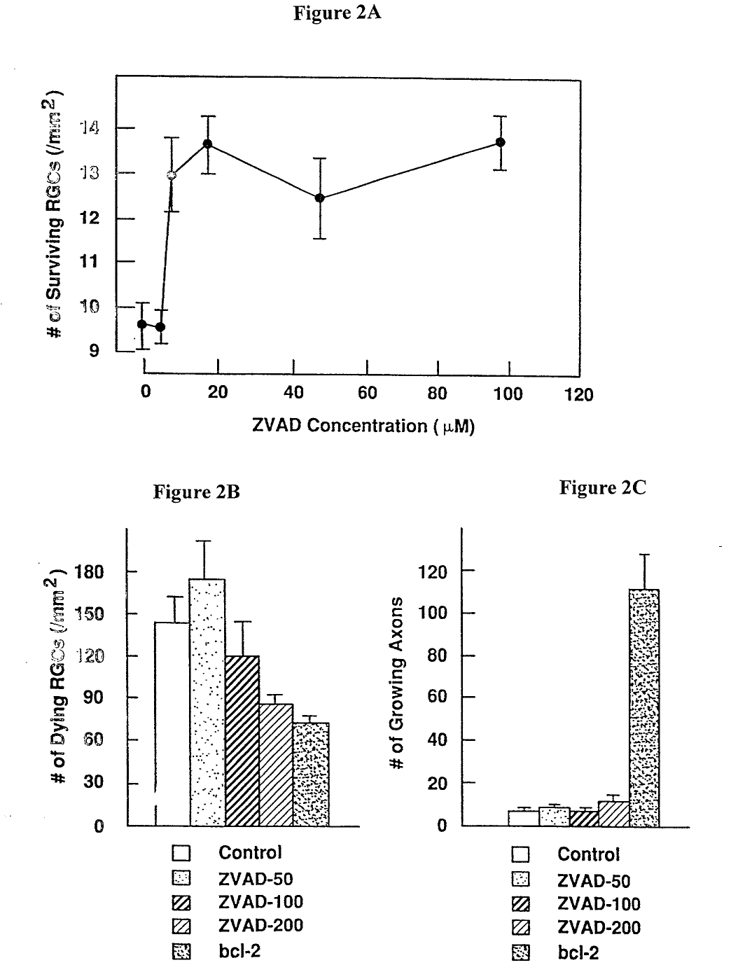

Whether these two activities of neurons, survival and axonal growth, can be separated in vitro was next examined. The anti-apoptotic function of bcl-2 is well established (Davies, A. A TINS 18, 355-358 (1995); Korsmeyer, S. J. hnmunol. Today 13, 285-288 (1992); Farlie, et al. Proc. Natl. Acad Sci. USA 92, 4397-4401 (1995); Bonfanti, L. et al. J Neurosci. 16, 4186-4194 (1996)). Therefore, it is especially important to examine whether its growth-promoting activity is simply an indirect consequence of supporting cell survival. It has been suggested that bcl-2 suppresses apoptosis by impairing the activity of interleukin 1-converting enzyme (ICE) (Gagliardini, V. et al. Science 263, 826-828 (1994); Miura et al. Cell 75, 653-660 (1993), a cysteine protease implicated as essential in the process of cell death in vertebrates (Gagliardini, V. el al. Science 263, 826-828 (1994); Miura et al. Cell 75, 653-660 (1993); Henkart et al. Immunity 4, 195-201 (1996); Nicholson, D. W. el al. Nature 376, 37-43 (1995). Use of a chemical that blocks ICE activity, presumably the same pathway that bcl-2 uses to suppress 1s apoptosis, allowed testing of the relationship between the functions of axonal growth and cell survival. The capacity of an irreversible ICE-like protease inhibitor—ZVAD (Z-Val-Ala-Asp-CH₂F, Enzyme Systems Products) was investigated (Henkart, P.A. Immunity 4, 195-201 (1996); Nicholson, D. W. el al. Nature 3 76, 3 743 (1995); Fletcher, D. S. et al. J. Interferon Cytokin Res. 15, 243-248(1995)—to influence the outgrowth of retinal axons. Using a dissociated cell culture system that allows visualization of single cell morphology, cultures were prepared from retinae of P2 pups. RGCs were prelabeled by injecting DiI into the tectum of PO pups. Treatment with ZVAD at a concentration of 10 mM or above effectively reduced RGC death after 2 days in culture. Nevertheless, labeled RGCs from wild-type animals were round and devoid of neurites in culture (n=36), whereas, RGCs derived from bcl-2 transgenic mice (n=24) exhibited extensive axonal outgrowth. Note that this occurs in the absence of any neurotrophic factors added to the culture medium. The effect of the ICE inhibitor was also tested in the explant coculture system with tissue prepared from wild-type P2 mice. Treatment with ZVAD reduced the extent of cell death in retinal explants (n=22) (FIG. 2A). A concentration of 200 mM of ZVAD protected cells from death almost as well as bcl-2 in the transgenic mouse (n 6); however, the number of axons that invaded tectal slices was 10-fold less in cultures from wild-type animals than in those from bcl-2 transgenic mice (n=22) (FIG. 2B). While treatment with ZVAD was sufficient to prevent death of RGCs, it is not sufficient to promote axonal growth. By increasing the concentration of ZVAD, the number of dying cells in retinal explants decreased, whereas, the number of growing axons did not change significantly. Therefore, these examples suggested that cell survival and axonal growth are two distinct activities of RGCs; bcl-2, but not ICE inhibitors, supports both of these activities. Evidence from other investigators (when viewed in conjunction with that provided herein) also support the theme that cell survival and axonal growth are two independent activities of neurons (Sagot, Y., Tan, S. A-Hammang, J. P., Aebischer, P. & Kato, A. C. J Neurosci. 16, 2335-2341 (1996); Dusart, 1. & Sotelo, C. J Comp. Neurol. 347, 211-232 (1994)). The regenerative failure of retinal axons and the decrease of bcl-2 levels in RGCs occur (E 18) before programmed cell death starts (P 1 P5) (Young, R. W. J Comp. Neurol. 229, 362-373 (1984)). The dissociation supports the observation from other investigators that the expression pattern of bcl-2 does not mirror recognized patterns of cell death in the CNS (Merry, D. E., Veis, D. J. Hickey, W. F. & Korsmeyer, S. J. Development 120, 301-311 (1994)); instead, it appears to correlate with cell differentiation and capacity for axonal outgrowth of neurons. Second, before programmed cell death begins, cell counts from spinal and facial motor neurons showed no significant difference in bcl-2 knockout mice and in wild-type animals (Michaelidis, T. M. et al. Neuron 17, 75-89 (1996)) whereas, a drastically reduced number of growing axons in cultures from bcl-2 knockout mice was found. Third, the ZVAD experiments further demonstrated that ICE inhibitor, though sufficient to block cell death, is not sufficient to support axonal growth. These all support the position that bcl-2 promotes axonal growth through a mechanism independent of its anti apoptotic activity.

### Example 6

### Materials and Methods for Examples 1-5

Retinotectal Cocultures

Brains were dissected into ice-cold Gey's balanced salt solution enriched with glucose. Coronal slices through the superior colliculus were cut with a McIIwain tissue chopper at a thickness of 300 μm. Retinal explants were abutted against tectal slices. Tissues were placed on the microporous membrane of Millicell wells (Millipore) and maintained in NeuralBasal medium supplemented with B27 (GIBCO Inc., New York) at 370C for five days. To exclude the possibility that tectal tissues from mutant mice may affect axonal growth of RGCs, a series of parallel experiments were performed in which one retinal explant of each mouse was confronted with the tecturn from the same mouse, while the second retinal explant was placed against the tecturn from another mouse. With this arrangement, retinal explants from each animal had the possibility of being cocultured with the tecturn from a wild-type, heterozygous, or homozygous animal. The number of regenerating axons was sampled by applying the lipophilic carbocyanine fluorescent label, DiI, in crystals to fixed retinal explants. The cocultures were stored in fixative for two-four weeks to allow diffusion of the dye, and labeled retinal axons were viewed with a fluorescence microscope (Nikon).

Mouse pups were obtained from matings of males heterozygous for the bcl-2 transgene with C57BL/6J females. Four days after birth (P4), pups received a unilateral transection of the optic tract at the mid-tectal level. Regeneration of the optic tract was assessed using anterograde tracing with CT-13 (cholera toxin B), ten days after nerve transection. To visualize the axons, a diaminobenzidine (DAB) color reaction was carried out using a slightly modified version of the protocol of Angelucci, et al (Angelucci, A., Clascd, F. & Sur, M. J. Neurosci. Meth. 65, 101-112 (1996)). In brief, brains were cut into 50 in sagittal sections; every other section of the brain was collected for cresyl violet staining, and the other section was incubated with primary antibody against CT-B at 4C for 96 hr and then further processed with ABC elite kit (Vector).

The brain sections were visualized with a Nikon microscope and site of the lesion was reconstructed in 3 dimensions with MIT Neurotrace computer software. Primary cultures of dissociated retinal cells were prepared from P21 wild type or transgenic animals. RGCs were prelabeled by injecting DiI solution (25% in Dimethyl Formamide) into the tectal region bilaterally in PO pups. Cells were plated in 24-cell wells treated with poly-L-lysine (10 μg/ml, 4° C. overnight) and coated with Human Merosin (0.2 g/ml, r.t., 2 hr)(Meyer-Franke, A. and Barres, B. A. Neuron 15, 805-819 (1995)). Cultures were maintained for 2 to 3 days in NeuralBasal medium supplemented with B27. Trypan blue staining was used to examine the viability of retinal ganglion cells (RGCs). Retinotectal cocultures prepared from wild-type P2 mice were described previously and ZVAD (Z-Val-Ala-Asp-CH₂F, Enzyme Systems Products) was added to the culture medium at the time of plating. Cell death was detected by staining with the fluorescent dye, SYTOX green fluorescent dead cell stain (Molecular Probes). Cultures were visualized under an inverted Nikon microscope equipped with Nomarski and epifluorescence illumination.

Immunoflorescent staining. For immunofluorescence staining of bcl-2, embryonic day 16 or 18 (E 16 or E 18) embryos were obtained by Caesarian section of timed mated wild type mothers. Brains were removed and fixed in 4% paraformaidehyde overnight and cut into transverse sections of 10 μm thickness with a cryostat. Sections were blocked with PB S containing 2.5% normal goat serum, 2.5% fetal bovine albumin, and 0.3% Triton X-100 for 30 min. at room temperature, and then incubated with affinity purified primary antibody (hamster anti-mouse bcl-2, 1:50, PharMingen) at 4° C. overnight. Secondary antibody (FITC-conjugated goat antibody to hamster immunoglobulin, 1:200) was then applied to the slide for 2 hr at room temperature. The slides were rinsed several times in PBS, mounted in Fluoremount G and viewed with the fluorescence microscope.

### Example 7

### Overexpression of Bcl-2 Supports Robust Optic Nerve Regeneration in P3=Mice

Our strategy was to perform optic nerve crush in P3 mouse pups, long before myelination had begun, while the CNS environment was likely to be permissive for axon regeneration (details of the methods are provided in Example 13). To enable the visualization of axons immediately after optic nerve injury, we injected anterograde tracer fluorescence-CTB into the eye right after the optic nerve surgery. In order to determine the labeling efficiency of intraocular injection of fluorescence-CTB, we examined 6 control mice that had received only surgery to expose the right optic nerve and CTB injection, and 4 mice with an incomplete optic nerve crush. We found that under both conditions, fluorescence-CTB was transported rapidly, passing beyond the point of the optic chiasm and entering the brain area within 24 hr of surgery. Within 48 hr of injection, fluorescence-CTB labeled the entirety of their brain target areas—the lateral geniculate nuclei (LGN) and SC. Thus, fluorescence-CTB labeled efficiently the entire pathway of healthy optic axons within 48 hr of dye injection. This knowledge helped us to identify incomplete optic nerve axotomy following the crush, in our later experiments.

To study optic nerve regeneration, we performed complete optic nerve crush followed by CTB injection. The surgical procedure and data analysis were carried out under littermate controls, without prior genotyping. The first group of mice (n=16) was sacrificed at 24 hr post surgery (1 DPO). After genotyping, 11 of these pups were determined to be wild-type and 5 Bcl-2 transgenic. No regenerative response was observed in any of the 11 wild-type mice that had received the optic nerve crush. Fluorescence-CTB labeling stopped proximal to the crush site, and no growth into or beyond the lesion was detected. Furthermore, many CTB-labeled axons revealed “crystalized” or “degeneration-like” swelling bulb morphologies along the path, an implication of ongoing axon degeneration. These results corroborate previous reports that the optic nerve degenerates rapidly following axotomy in early neonatal stages in mice (Miller and Oberdorfer, 1981; Allcutt et al., 1984a).

In Bcl-2 overexpressing mice, remarkably, we observed massive axon regeneration in all cases examined. At only 24 hr post surgery, large numbers of CTB-labeled axons were seen passing directly and rostral to the lesion, ending at 500-1,000 μm beyond the lesion. No fluorescence labeling was found beyond this point of the optic nerve sheath, nor in the brain sections. This indicated that the fluorescence-CTB labeling revealed regenerating axons but not fibers spared from incomplete crush; otherwise, the CTB labeling would be seen passing the optic chiasm into the brain sections at this time point. Unlike in the wild-type mice, labeled axons appeared healthy and fasciculated, resembling the morphology of elongating axons during early development.

To corroborate the observations following CTB labeling, we then performed GAP-43 and NF-L immunostaining. Since anti-GAP-43 was unable to stain degenerating axons, it failed to label optic nerve fibers in the nerve sheath of injured wild-type mice, either proximal or distal to the lesion. The staining results confirmed rapid axonal degeneration induced by optic nerve crush in neonatal wild-type mice. In contrast, the optic nerve sections from Bcl-2 transgenic mice revealed strong GAP-43 labeling of axon fascicles ending at 500-1,000 μm beyond the lesion. The labeling pattern was identical to that revealed by fluorescence-CTB. Confocal microscopy revealed that anti-NF-L weakly stained axon fragments and degenerating fibers in wild-type optic nerve sections. The anti-NF-L staining in Bcl-2 transgenic mice, however, was very strong, with many revealing bullet-shaped, growth cone-like structures at their ends, an indication of characteristic, active axonal growth. Thus, regenerating axons were labeled rather than those spared from incomplete optic nerve crush, leading to the conclusion that overexpression of Bcl-2 is sufficient to promote robust optic nerve regeneration in P3 mice.

### Example 8

### Overexpression of Bcl-2 Supported Long Distance Axon Regeneration and Target Recognition

Nerve regeneration in vivo involves not only neuronal survival and successful initiation of axon elongation following injury, but also the ability of regenerating axons to extend over long distances and follow the correct positional cues on their path to locate and reconnect with their original targets. To further determine the fate of regenerating axons, subsequently, we studied mice at 2 and 4 DPO (Table 1).

TABLE 1
Summary of CTB- and FluoroGold-Labeling Results

| | Total % of | | | | |
|---|---|---|---|---|---|
| | Positive | % with CTB-labeling | | | % with FG |
| Genotypes | Labeling | 1 DPO | 2 DPO | 4 DPO | Labeling |
| Wild-type | 2.4% | 0% | 0% | 5.3% | 0 |
| | (n = 42) | (n = 11) | (n = 4) | (n = 19) | (n = 8) |
| Bcl-x_{L} transgenic | 7.1% | 11.1% | N/A | 0 | N/A |
| | (n = 14) | (n = 9) | | (n = 5) | |
| Bcl-2 transgenic | 96.4% | 100% | 100% | 91.7% | 100% |
| | (n = 28) | (n = 5) | (n = 5) | (n = 12) | (n = 6) |

Percentages of mice with positive CTB-labeling of optic nerve fibers past beyond the crush site or positive FluoroGold labeled RGCs in their retinas. The value is obtained by the number of mice with positive CTB or FluoroGold (FG) labeling divided by the total number of mice examined at that particular time point. Data are collected at 1, 2, and 4 DPOs for CTB-injected mice or at 11 DPO for mice with FluoroGold labeling.

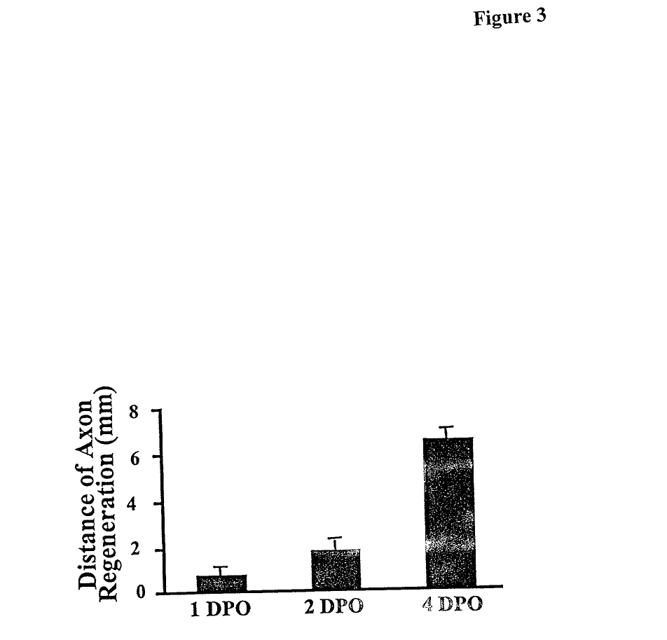

Fluorescence-CTB and GAP-43 immunofluorescence labeling for optic nerve sections sampled at 2 DPO both yielded results similar to those seen at 1 DPO. Both revealed degenerating axons and the lack of regeneration in the wild-type mice (n=4); whereas, in Bcl-2-overexpressing mice, labeled axons formed fascicles and extended beyond the lesion site, up to 1,000-2,000 μm across the lesion, or 0.5-1.5 mm further than measured at 1 DPO (n=5) (FIG. 3).

At 4 DPO, a total of 31 mice were studied. After genotyping, 19 were determined to be wild-type and 12 Bcl-2 transgenic. In all but 1 of the wild-type mice, severed axons degenerated completely, and no fluorescence-CTB labeling was observed in the optic nerve sheath or along the retinofugal pathway in the brain. In this case only, the entire optic nerve and its pathway in the brain were labeled, and a crush site could be clearly identified. Based on the knowledge that severed optic nerves of postnatal wild-type mice never regenerate, we determined that this was a result of incomplete nerve damage. As there was only 1 surgery out of 19 performed on wild-type mice yielded incomplete damage, the failure rate for complete nerve damage using our procedure was quite low. In contrast, in 11 of the 12 Bcl-2-overexpressing mice analyzed, intensively labeled regenerating axons filled the entire optic nerve sheath, passing through the optic chiasm, as revealed by fluorescence-CTB and anti-GAP-43 labeling. In only 1 of the Bcl-2-overexpressing mice, did labeled optic nerve fail to show any sign of regeneration and appear to be degenerated completely.

To determine whether the regenerating axons could find the appropriate pathways and locate their original targets, we examined their brain sections. Brain sections were cut sagittally, and labeled axon trajectories were reconstructed in 3-dimensions. As expected, no fluorescence-CTB was found in brain sections of the wild-type mice from 1-4 DPO, except in the single case of incomplete nerve crush. In Bcl-2 overexpressing mice, fluorescence-CTB was also negative in the brain sections collected at 1 (n=2) and 2 (n=3) DPO, consistent with the observation that regenerating axons had not passed the point of optic chiasm at this stage. In contrast, at 4 DPO, extensive fluorescence labeling was found in the contralateral brain sections of Bcl-2 overexpressing mice, revealing regenerating axons traveling in the optic tract and covering most of their original targets, the LGN and SC (n=6). Ipsilateral labeling of regenerating axons was also detected but was much less dense than the contralateral. The ipsilateral labeling was seen primarily in the small segregated area of LGN that is normally innervated by the ipsilateral retinal projections. Given that there is a greater number of contralateral RGC projections than ipsilateral projections in normal mice, the distribution of RGC projections and the pattern of the CTB labeling were almost indistinguishable from that in the non-crushed control mice.

To determine whether the regenerating axons could remain connected with their targets after a long-term survival, we studied mice at 30 DPO. Positive CTB labeling was again found in the LGN and SC of Bcl-2-overexpressing mice (n=3) but not in those of wild-type controls (n=3).

Taken together, we conclude that overexpression of Bcl-2 supports long distance optic nerve regeneration, and that regenerating axons are able to locate the appropriate targets and re-establish the topographic map at this stage.

### Example 9

### Overexpression of Bcl-2 Induces Rapid Axon Elongation

An important measure for the intrinsic capacity of axon regeneration is the speed of their elongation. Mature RGCs are known to extend axons at a much slower speed than are the embryonic RGCs (Jhaveri, S. et al. (1991), Exp. Brain Res. 87:371; Goldberg, J. L. et al. (2000), Annu. Rev. Neurosci. 23:579). To determine the rate of axon regeneration in Bcl-2-overexpressing mice, we calculated the growth rate of regenerating axons by subtracting the distance of axon regeneration measured at 2 DPO (1.0-2.0 mm) from that at 1 DPO (0.5-1.0 mm) and obtained a rate of 0.5-1.5 mm/day. Using another method to calculate the rate of axon regeneration, we also measured the full length of the optic nerve and added it to the travelling distance of the optic tract, measured from the optic chiasm to the posterior border of the SC in P7 mice. The total distance measured was 6-8 mm, and thus, the resultant average speed of axonal regeneration (to reach the SC in 4 days) was 1.5-2 mm/day, which compares favorably to the calculated speed of axonal extension from 1 to 2 DPO. Taken together, we determined that Bcl-2 induced optic nerve regeneration extended at a rate of 0.5-2 mm/day. This is equivalent to the rate of axonal elongation during embryonic stages (Jhaveri et al. (1991), Exp. Brain Res. 87:371-382; Collelo et al. (1992), J Comp Neurol 317:357-378; Goldberg J L, Barres B A (2000); Annu Rev Neurosci 23:579-612). Thus, overexpression of Bcl-2 prevents the maturational loss of the intrinsic regenerative capacity of RGC axons.

### Example 10

### A Majority of RGC Axons Regenerate in Bcl-2-overexpressing Mice

If Bcl-2 is responsible for maintaining the intrinsic regenerative capacity of RGC axons, in Bcl-2-overexpressing mice, the majority of RGCs should regain their ability to regenerate axons in neonatal stages, in a favorable environment. To corroborate this, we quantitated the number of RGC axons induced to regenerate and successfully reached their brain targets in Bcl-2-overexpressing mice. This was made by placing a retrograde tracer, FluoroGold, in the RGCs' most distant target—the SC—following optic nerve crush or in age-matched non-crushed control mice. Thus, RGCs with regenerated axons or axons connected to the SC would be labeled retrogradely. Our previous studies have indicated that regenerating axons of Bcl-2-overexpressing mice reached the SC at 4 DPO. Then, it took 6-7 days for FluoroGold to be fully transported from the SC to RGCs. We, therefore, sacrificed mice at 11 DPO and counted the numbers of retrogradely labeled RGCs in the retinas. FluoroGold labeled numerous RGCs as well as microglia in uninjured eyes. RGCs are normally round or oval cells, while microglia are characteristically spiny cells with bright fluorescence and multiple cellular processes (Thanos et al. (1993) J Neurosci 13:455-466).

In our study, these two types of cells could be readily distinguished by their morphologies and their locations in different optic planes. We found that in uninjured eyes, the counted RGC density was 1,110.9±309.7 cells/mm² for wild-type mice (n=5) and 2,102.1+426.9 cells/mm² for Bcl-2-overexpressing mice (n=5). Because Bcl-2 prevented the developmental loss of RGCs, the retinas of Bcl-2-overexpressing mice normally contained a higher number of RGCs than those of wild-type mice.

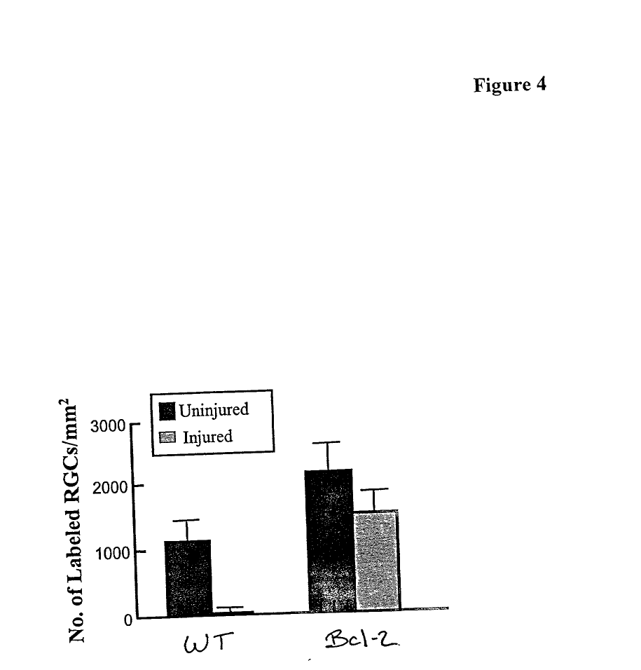

In retinas of wild-type mice that underwent the optic nerve crush, we detected very few labeled round or oval cells, presumably RGCs, due to the lack of regeneration (34.2 +24.1 cells/mm²; n=4). At the same time, small spiny cells, identified as active microglia, did appear. In contrast, numerous cells in the retinas of Bcl-2 transgenic mice were strongly labeled. We obtained a cell density of 1478.4±328.4 RGCs/mm² in their whole-mount retinas (n=6), equivalent to 70% of the RGC density of uninjured Bcl-2 retinas (FIG. 4). Thus, we confirmed the robust regeneration of the severed optic nerves with overexpression of Bcl-2. The majority of RGCs from Bcl-2 transgenic mice were able to reconnect with the SC after injury, while 100% of those from wild-type mice failed to do so under similar conditions.

It is unlikely that our data could be explained by incomplete axotomy of the optic nerve. First, transgenic mouse pups appeared normal and could not be distinguished from their wild-type littermates phenotypically. All surgical procedures and analysis were performed prior to genotyping. Second, in every case, we were able to identify the crush site by visual inspection of the optic nerve and by a traumatized zone that contained degenerated cells and tissue debris in immuno- and histo-chemical stained optic nerve sections. There were total of 42 wild-type mice received P3 optic nerve crush and 31 wild-type and Bcl-2-overexpressing mice received P5 optic nerve crush, only 1 of which exhibited any finding that might be consistent with incomplete nerve crush. Nevertheless, following P3 optic nerve crush, 27 out of 28 of Bcl-2 overexpressing mice exhibited clear evidence of axon extension far beyond the crush site. Finally, the fact that CTB-fluorescence labeled axons were seen to extend for various distances caudal to the lesion at 1-4 DPO supports the conclusion that we observed regenerating axons in Bcl-2-overexpressing mice, but not those sparing from incomplete axotomy. Uncut, spared axons would be expected to extend through the entire optic nerve and be observed in the brain within 24 hr following optic nerve damage. Moreover, numerous growth cones, observed only in the Bcl-2 transgenic mice, provide a strong indication for active fiber regeneration and should not be observed with spared axons.

A recent study has reported that overexpression of Bcl-2 fails to promote optic nerve regeneration if the injury is incurred at P5 (Lodovichi et al. (2001) Eur. J. Neurosci. 13:833). We therefore performed the optic nerve crush on P5 Bcl-2-overexpressing mice and assessed the regeneration at 10 and 30 DPO.

A total of 31 mice were studied. After genotyping, 15 were determined to be wild-type and 16 Bcl-2 transgenic. In all cases studied, we found no positive CTB-fluorescence labeling in their brain sections examined, regardless they were from wild-type or Bcl-2-overexpressing mice or were sacrificed at 10 or 30 DPO, indicating a failure of optic nerve regeneration into the brain. When the optic nerve sections were examined, degeneration of severed optic nerves in wild-type mice was clear. No labeling of CTB-fluorescence or anti-NF-L immunofluorescence was observed. In those of Bcl-2-overexpressing mice, although a few fibers were present at 10 DPO, no signs of regeneration were discovered. All surviving fibers stopped anterior to the crush site. Thus, our findings are in agreement with those of Lodovichi et al. (2001), supra, that optic nerve regeneration is blocked in Bcl-2-overexpressing mice if the injury is incurred at P5.

Thus, these results demonstrate elongation to long distances and to the rights cues. The results also show that loss of the intrinsic regenerative capacity by mature CNS axons is a major impediment to successful CNS regeneration in vivo, and that overexpression of Bcl-2 is sufficient to support the intrinsic regenerative potential of CNS axons. The results show a novel role of Bcl-2 in regulating CNS regeneration, unrelated to its control of apoptosis. The results also show the successful navigation of the optic tract and arrival in appropriate target fields by the regenerating axons of Bcl-2 transgenic mice demonstrate that if axon growth can occur, reconstitution of normal circuitry will result, at least in the neonatal period. Finally, our results implicate two parallel mechanisms in the modulation of CNS regeneration: loss of intrinsic growth potential by CNS axons and appearance of inhibitory molecules in the environment. Thus, Bcl-2 transgenic mice, whose neurons retain the intrinsic capacity to regenerate axons, provide a unique model for defining inhibitory mechanisms in the CNS environment.

We have shown herein that overexpression of Bcl-2 is sufficient to promote optic nerve regeneration in vivo, if the damage is incurred at P3, before optic nerve myelination begins (Jhaveri et al. (1992) Glia 6:138-148; Demerens et al. (1996) PNAS 93:9887-9892). We also confirmed the failure of optic nerve regeneration in Bcl-2 transgenic mice if nerve injury is incurred at P5 (unpublished results), coinciding with the onset of optic myelination (Foran et al. (1992) J. Neurosci. 12:4890; Demerens et al. (1996) Proc Natl Acad Sci U S A. 93:9887). These results, together with many other reports, suggest that myelin/oligodendrocyte maturation plays a role in blocking optic nerve regeneration, even if neurons are intrinsically capable of re-initiating axon growth (for review, see Schwab et al. (1993), Annu Rev Neurosci 16:565-595, Fournier et al. (2001) Curr. Opin. Neurobiol. 11:89). Our results suggest that optic nerve regeneration in postnatal mammals is determined by two independent mechanisms: an intrinsic change of regenerative capacity of RGC axons and maturation of a non-permissive environment (Fawcett J W (1992), Trends Neurosci 15:5-8; Schwab et al. (1993) Annu Rev Neurosci 16:565-595; Holm K, Isacson O (1999) Trends Neurosci 22:269-273). That same concept may apply to adult mice has been clearly demonstrated in our retino-brain slice co-culture experiments. Even in adults, retinal explants from Bcl-2 transgenic mice readily regenerated their axons into embryonic brain environment but failed to invade mature brain slices. Studies of retinal axon maturation in rodents have revealed two distinct stages of axon growth—elongation (at an immature stage) and arborization (later in development)—distinguished in part by contrasting rates of axon extension (Holm K, Isacson 0 (1999) Neurosci 22:269-273; Jhaveri et al. (1991) Exp Brain Res 87:371-382; Goldberg J L, Barres B A (2000) Annu Rev Neurosci 23:579-612). Embryonic RGC axons elongate at a speed about 10 times faster than mature ones (Collelo and Guillery (1992) J Comp Neurol. 317:357). This difference has been attributed primarily to the maturational change in the intrinsic property of neurons (Davies A M (1989) Nature 337:553-555). Our current data indicate that overexpression of Bcl-2 restores the growth rate of regenerating axons of postnatal RGCs to values characteristic of embryonic life. Therefore, Bcl-2 is a potent regulator of the growth potential of RGCs.

Our results strongly suggest that Bcl-2 supports axon regeneration via a novel mechanism that is unrelated to its control of apoptosis. As described below, Bcl-x_{L} does not significantly stimulate axon regeneration, even though Bcl-x_{L} is the closest anti-apoptotic member of the Bcl-2 family, and the two are thought to share common pathways in regulating apoptosis (Gonzalez-Garcia et al. (1995) Proc Natl Acad Sci U S A 92:4304-4308). Comparison between signaling pathways induced by Bcl-2 and Bcl-x_{L} expression, such as DNA microarray, may provide important clues about the underlying mechanisms of these two proteins in their regulation of cell survival and nerve growth. It has been shown that overexpression of Bcl-2 in PC12 cells accelerates neuronal differentiation and polymerization of neurofilament proteins (Suzuki A, Tsutomi Y (1998) Res 801:59-66). A plausible mechanism may be that Bcl-2 interacts with the signal transduction pathways that regulate neural differentiation, e.g., Ras and Raf, to support axonal growth (Femandez-Sarabia M J, Bischoff J R (1993) Nature 366:274-275; von Gise A et al. (2001) Mol Cell Biol 21:2324-2336). Indeed, emerging evidence now indicates that elevation of Bcl-2 expression affects mitogen-activated protein (MAP)-kinase activity, which is thought to be a key enzyme of the signal transduction cascade for neural differentiation and neurite extension (Katoh et al. (1999) Biochem J 338:465-470). In any case, our findings suggest that stimulating the intrinsic growth potential of mature CNS axons will be an essential component of the strategy to achieve full CNS regeneration.

### Example 11

### Bcl-x_{L}, Blocks RGC Death

The most well-established function for Bcl-2 is the regulation of apotosis (Dubois-Dauphin et al. (1994), Proc Natl Acad Sci U S A 91:3309-3313; Farlie et al. (1995) Proc Natl Acad Sci U S A 92:4397-4401; Bonfanti et al. (1996) J Neurosci 16:4186-4194; Bonfanti et al. (1996) J Neurosci 16:4186-4194). An intriguing question would be whether Bcl-2 affects the intrinsic mechanisms of axon regeneration directly or, simply, promotes neuronal survival with regeneration occurring as a default mechanism of mature neurons. The most direct way to address this question would be to compare the effect of Bcl-2 with that of another anti-apoptotic member of the Bcl-2 family, such as Bcl-x_{L}, which shares a common mechanism with Bcl-2 to support cell survival, for review, (see Reed et al. (1997), Nature 387:773). We, thus, studied mice overexpressing Bcl-x_{L} under the control of a strong neural promoter (Parsadanian et al. (1998) J. Neurosci. 18:1009).

First, we examined whether overexpression of Bcl-x_{L} is sufficient to prevent RGCs from nerve injury-induced cell death and if it is as effective as Bcl-2 overexpression. We compared the survival of RGCs following optic nerve crush in wild-type mice and mice overexpressing Bcl-2 or Bcl-x_{L} which were backcrossed to the same genetic background (C57BL/6J). High levels of Bcl-x_{L} or Bcl-2 transgene expression in the retinas of P2-5 transgenic mice were confirmed using western blot and reverse transcription polymerase chain reaction.

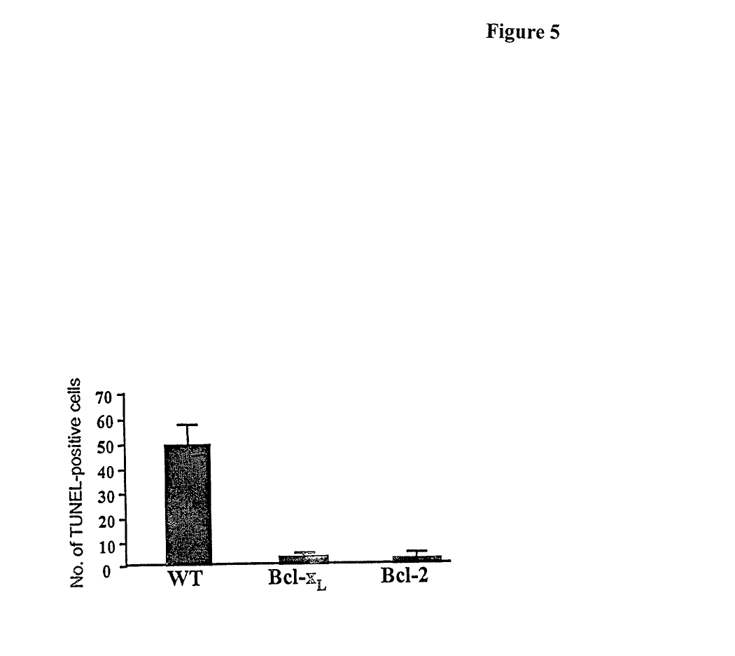

Since the peak period of RGC degeneration following neonatal optic nerve damage has been shown to occur at 24 hr post surgery (1 DPO), (Bonfantiet al. (1996) J Neurosci 16:4186-4194), we performed optic nerve surgery on P3 mice and examined RGC survival at 1 DPO. Using Cresyl Violet stain to reveal live RGCs, we found that, in the absence of optic nerve damage, retinas of mice of all genotypes exhibited multiple cell layers in the ganglion cell layer (GCL) at P4. The number of cells in the GCLs of wild-type mice was 622±166 (n=5) per retinal section. In Bcl-x_{L}- and Bcl-2-overexpressing mice, the numbers were 1130±220 (n=4) and 1037±198 (n=3), respectively. The higher numbers of RGCs in the GCLs of Bcl-x_{L}- and Bcl-2-overexpressing mice indicate that Bcl-x_{L} blocked developmental cell death of RGCs, as effectively as Bcl-2 did. At 24 hr after optic nerve crush, many RGCs from wild-type mice degenerated and displayed pyknotic profile, consistent with our observation that neurodegeneration occurred rapidly in wild-type mice following P3 optic nerve crush. The number of RGCs counted from the GCLs of wild-type mice was 379±67 (n=2) per retinal section, representing a 39% cell loss following injury. Most RGCs of Bcl-x_{L}- and Bcl-2-overexpressing mice, however, survived the optic nerve crush. The numbers of their RGCs were 1101±195 (n=3) and 943±162 (n=3), respectively, representing a 3% and 10% RGC loss. In addition, few cells revealed apoptotic morphology in the retinas of transgenic mice. Using in situ labeling of TUNEL to stain apoptotic cells, we confirmed the robust effect of Bcl-x_{L} and Bcl-2 in supporting RGC survival. By counting TUNEL-positive cells in the GCL, we obtained 50.3±8.5 (n=4) positive-cells per retinal section of wild-type mice, 2.7±0.6 (n=3) of Bcl-x_{L}-overexpressing mice, and 1.8±0.9 (n=4) of Bcl-2-overexpressing mice (FIG. 5).

Overexpression of Bcl-x_{L} and Bcl-2 not only prevented RGC loss but also blocked the retrograde degeneration of injured optic nerve fibers. Staining with GAP-43 and neurofilament antibody revealed that the optic nerve fiber layer of the wild-type mice had largely degenerated; whereas, the nerve fiber layers in retinas of Bcl-x_{L}- and Bcl-2- overexpressing mice remained intact and were strongly labeled. Therefore, overexpression of Bcl-x_{L} blocked both axotomy-induced RGC death and nerve fiber degeneration, as effectively as Bcl-2.

### Example 12

### Bcl-x_{L} Failed to Promote Optic Nerve Regeneration in vivo and in vitro

We asked if Bcl-x_{L}, like Bcl-2, promoted optic nerve regeneration in neonatal mice. We showed previously that the expression of Bcl-2 was down-regulated in RGCs as they lose the intrinsic ability to regenerate axons: however, Bcl-x_{L} expression has been shown to remain high in RGCs throughout life (Levin et al. (1997) Invest Ophthalmol Vis Sci 38:2545-2553). We, therefore, hypothesize that Bcl-x_{L} may not be involved in the regulation of RGC axon regeneration.

Using similar method of axon tracing, we investigated optic nerve regeneration in Bcl-x_{L}-overexpressing mice at P3. We found that in contrast to the robust regeneration observed in Bcl-2-overexpressing mice, in 8 out of the 9 Bcl-x_{L} transgenic mice studied at 1 DPO (Table 1), fluorescence-CTB revealed a complete failure of regeneration. Labeling of axons stopped proximal to the crush site but not beyond it. Unlike in wild-type mice, in Bcl-x_{L}-overexpressing mice, labeled axons that failed to regenerate appeared healthy with no detectable sign of axon degeneration. In 1 of the 9 Bcl-x_{L} mice examined, CTB-labeling was found to extend 200 μm rostral to the lesion site (Table 1). GAP-43 immunostaining confirmed that, in 8 out 9 Bcl-x_{L}-overexpressing mice, the optic nerves stained positive for GAP-43 proximal to the crush but not beyond. The bullet-shaped, growth cone-like structures that were seen in the transected optic nerves of Bcl-2-overexpressing mice were not found here in most of the injured optic nerves of Bcl-x_{L} transgenic mice. We also examined optic nerve regeneration at 4 DPO. In 5 Bcl-x_{L} transgenic mice examined, similarly, no fluorescence-CTB labeling was seen in optic nerves distal to the crush site or in their brains (Table 1). These data verify the protective effect of Bcl-x_{L} on axonal degeneration without promoting regeneration.

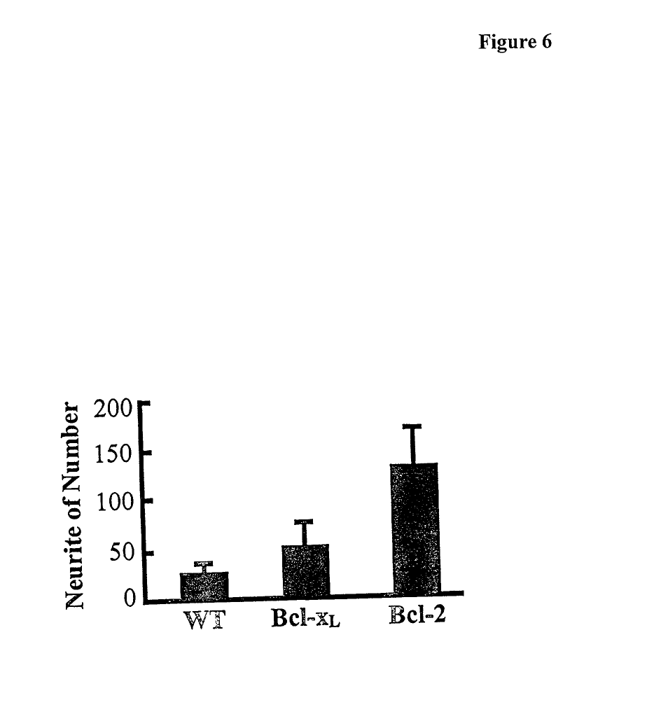

To further confirm the in vivo observation that Bcl-x_{L} did not promote optic nerve regeneration, we performed another set of experiments, using retino-tectal co-cultures prepared from tissues of P2 wild-type mice, and Bcl-x_{L}- or Bcl-2-overexpressing mice. Retinal explants derived from these mice were placed against brain slices that contained the area of SC. Because there were no axonal connections between the retina and brain slices when they were first placed in culture, the number of retinal axons that invaded the brain slices after incubation reflected regenerative activity of retinal axons. We discovered that retinal explants derived from both wild-type mice and mice overexpressing Bcl-x_{L} exhibited low levels of axon regeneration (FIG. 6). The number of labeled retinal axons that invaded brain slices was 23.8±11.2 (n=10) and 50.0 ±27.8 (n=5). In contrast, extensive neurite outgrowth was observed from co-cultures prepared from Bcl-2-overexpressing mice, as described above; the number of regenerating axons was 140.3±42.7 (n=8). There was a slight increase in the number of axon regeneration in cultures prepared from Bcl-x_{L}-overexpressing mice in comparison to those of wild-type mice. This increase might be due, in part, to the improvement of cell survival with Bcl-x_{L} overexpression. It is reasonable to conclude that Bcl-x_{L}, although supports the survival of RGCs following nerve damage, cannot promote optic nerve regeneration.

### Example 13

### Materials and Methods for Examples 7-12

Animals. Wild-type, Bcl-2, and Bcl-x_{L} transgenic mice were obtained from matings of wild-type C57BL/6J females with either males carrying the Bcl-2 transgene under the control of neuron-specific enolase promoter (line 73a), (Martinou et al. (1994) 13:1017-1030), or males carrying the Bcl-x_{L} transgene under the control of Tal (α-tublin) promoter (line 7193) (Parsadanian et al. (1998) J Neurosci 18:1009-1019. Thus, within each littermate, only half of the offspring would be Bcl-2 or Bcl-x_{L} transgenic, while the other half would be wild-type to serve as littermate controls. Both transgenic mouse lines were bred on a similar genetic background (C57BL/6J) to limit genetic variations. All experimental procedures were carried out without knowledge of genotype. Indeed, genotypes were not determined until mice were sacrificed. Genotypes were determined using a standard polymerase chain reaction methodology on tail DNA.

Optic nerve surgery and anterograde labeling of axons. The date of birth was designated postnatal day 0 (P0). Three or five days after birth, mouse pups were anesthetized by hypothermia. To allow axotomy of RGCs while preserving the optic nerve connective sheaths, which act as a scaffold for regenerating fibers, we used the optic nerve crush procedure (Chierzi et al. (1999) J Neurosci 19:8367-8376). The left optic nerve was exposed intraocularly and crushed with Dumont #5 fine surgical forceps for 12 seconds. The crush was performed about 1 mm from the posterior pole of the eyeball to avoid damaging the ophthalmic artery. Successful nerve damage was verified by visual inspection following sacrifice. Control pups received a similar operation to expose the optic nerve without crush.

To enable visualization of axons from day post operation (DPO), for some groups, an anterograde tracer, cholera toxin B subunit (CTB) conjugated with fluorescein (FITC) or rhodamine (RITC) (List Biological Lab, Inc.; Campbell, Calif.)(2.5 μg/μl in phosphate buffered saline [PBS]), was injected into the vitreous cavity immediately after the optic nerve crush. Other mice were allowed to survive for 10 or 30 days. Mice with longer-term survival were anesthetized again with 2.5% Avertin at 3 days before sacrifice; their right eye were injected with CTB-fluorescence. After 3 days, these mice were sacrificed for analysis.

Histology and immunofluorescence labeling. After 1-30 days post surgery, mice were anesthetized and transcardially perfused with PBS, followed by 4% paraformaldehyde in PBS. The eyecups, the entire optic nerve, and the brains were carefully dissected under a stereoscopic zoom dissection microscope and sectioned at 14 μm on a cryostat. Histochemistry (Cresyl Violet) and immunofluorescence detection of neuronal markers were carried out on adjacent sections. For immunofluorescence reaction, primary antibodies against growth-associated protein 43 (GAP-43) (Chemicon; Temecula, Calif.) and low molecular weight neurofilament protein (NF-L) (Chemicon) were used to reveal the morphology of axons. In brief, sections were blocked for 30 min at room temperature in A buffer: PBS containing 2.5% fetal bovine serum, 2.5% goat serum and 0.2% Triton X-100. Subsequently, they were incubated at 4° C., overnight, with primary antibody against either GAP-43 (1:400) or NF-L (1:500) in A buffer. Sections were then washed 3 times in PBS, incubated for 2 hr at room temperature with FITC-conjugated secondary antibody IgG (1:200, Chemicon) in A buffer, washed in PBS, and mounted with Vectashield mounting media (Vector; Burlingame, Calif.). The double labeling of immunofluorescence and CTB-fluorescence of optic axons were thus observed at lOx-40x objectives under a Nikon TE300 microscope equipped with fluorescence illumination.

Confocal Microscopy. The stained retina and optic nerve sections were examined with a Leica scanning laser confocal microscope system. The RITC fluorescence (red) was selectively excited with the 568 nm laser line and detected through a high-pass filter, RG 590. The FITC fluorescence (green) was excited with the 488 nm laser line and detected through an interferential narrow band filter centered at 535 nm (±8 nm). Tissue sections were viewed with a 100× magnification, 1.25 numerical aperture oil immersion objective. Fourteen to seventeen serial confocal planes at 0.1 μm intervals starting from the upper surface of each section were collected and formed into a stack image.

Evaluation of axon regeneration. Axon regeneration was evaluated under both a fluorescence and a confocal microscope, as described above. Positive regeneration was scored only if large numbers of labeled axons were seen to pass the lesion site, which was identified by a traumatized zone that contained degenerated cells and tissue debris. Both CTB-fluorescence labeling and anti-GAP-43 or anti-NF-L staining were used to confirm the results of axon regeneration. The results were scored blindly before mouse genotypes were known.

Quantification of cell survival To count viable RGCs, three Cresyl Violet-stained retinal sections that contained the optic nerve head were selected for each eye. All cells in the ganglion cell layer were counted in 7-11 fields (200 μm long) to cover the entire length of each section. Pyknotic cells were identified by the presence of condensed and darkly stained nuclei.

Quantification of cell death in the retinas was also assessed by TdT-mediated dUTP nick end labeling (TUNEL) reaction, using the in situ cell death labeling kit (Chemicon). Briefly, retinal sections prepared as described above were permeablized with 0.2% Triton-X 100 in 0.2% sodium citrate for 1 hr at room temperature, and incubated at 37° C. with TUNEL reaction mixture containing FITC-conjugated dUTP for 1 hr. The sections were then rinsed three times with PBS, mounted with Vectashield mounting solution, and observed under a Nikon fluorescence microscope. For each eye, 3 sections containing the optic nerve head were selected, and all TUNEL-positive cells in the RGC layer were counted. Results are presented as mean±SD.

Retrograde labeling of RGCs. P3 mouse pups were anesthetized by hypothermia and received a unilateral optic nerve crush, as described in the previous section. Immediately after the optic nerve surgery, a midline incision was made in the scalp above the superior colliculus (SC), and gelform (˜1 mm³, Upjohn; Kalamazoo, Mich.) soaked in FluoroGold solution (Fluorochrome; Denver, Colo.)(2% in PBS) was inserted over the colliculus. Ten days later, mouse pups were killed with an overdose of pentobarbital. Retinas were dissected, flat-mounted, and observed under the Nikon fluorescence microscope. Six standard rectangular regions (0.09 mm²), radically distributed at 1-2 mm from the optic nerve head, were photographed at 40× magnification by a cool color digital camera (Spot; Micro Video Instruments, Avon, Mass.) that was attached to the microscope and a Gateway GP6-300 computer. The location of the fields was chosen to avoid variations in RGC density as a function of distance from the optic disc. All of the FluoroGold-labeled cells in the photomicrograph that had morphologies resembling RGCs were counted. Counts were then averaged across the 6 regions. Again, the entire procedure was carried out prior to knowledge about mouse genotypes.

Retino-tectal co-culture. Retino-tectal co-cultures were prepared essentially as described above. Briefly, mouse pups were coded, anesthetized by hypothermia and sacrificed, and their tails were collected for genotyping. Mouse brains and retinas were then dissected, and coronal brain slices containing the SC were prepared with a McIIwain tissue chopper (Brinkmann; Westbury, N.Y.). Each retinal explant was placed to abut a brain slice on a 6-well cell culture insert, and cultures were maintained in Neurobasal medium (GIBCO; Grant island, N.Y.) supplemented with B27 (GIBCO) at 37° C. for 4 days. Subsequently, cultures were fixed with 4% paraformaldehyde in PBS for 1 hr, transferred to PBS, and stored at room temperature in the dark. Four crystals of 1,1′-dioctadecyl-3,3,3′,3′-tetramethylindocarbocyanine perchlorate (DiI)(Molecular Probes; Eugene, Oreg.) were placed onto each retinal explant. After allowing 2 weeks for dye diffusion, the number of retinal axons that had invaded the brain slices were counted under the inverted Nikon fluorescence microscope. Mouse genotypes were then decoded and data presented as mean±SD.

### Example 14

### Lithium Promotes Axon Regeneration In Retino-Brain Slice Co-Culture System

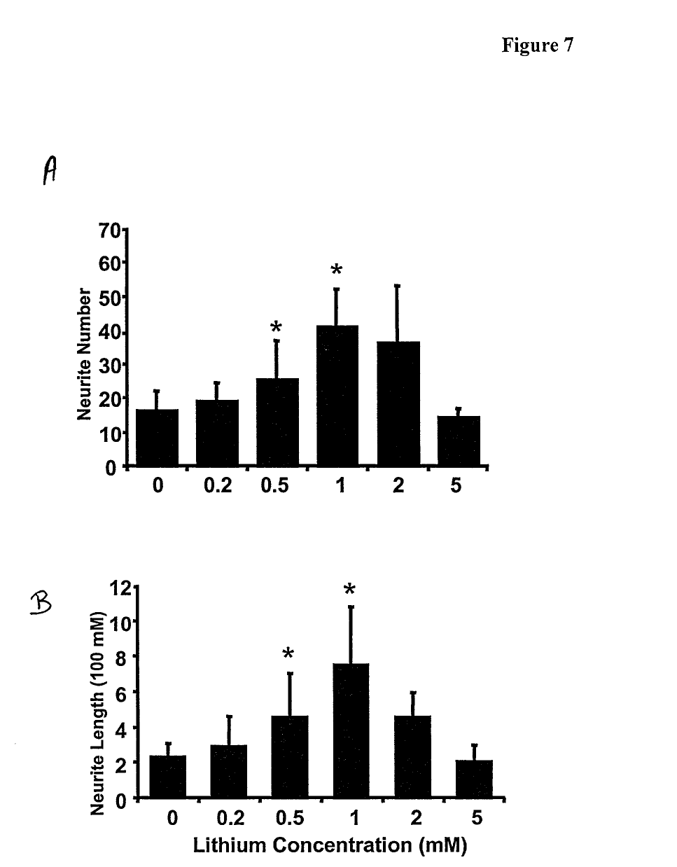

Taking advantages of the co-culture system described above, we investigated whether lithium supported the regeneration of RGC axons. Numbers and lengths of neurites extending from the retina into brain slices in the absence and presence of different concentrations of lithium chloride were quantified under a fluorescent microscopy (details of the methods are provided in Example 18). We found that in the absence of lithium chloride, cultured retinal explant derived from P2 wild-type mice grew average 18 neurites into brain slices, with average length of 220 μm (FIG. 7B). With increasing amounts of lithium chloride (0.1-5 mM), numbers and lengths of neurites that extended into brain slices increased in a dose dependent manner. The effect of lithium peaked at 1 mM with an average neurite number of 40 and 780 μm in length, representing ˜3 fold increase of axon regeneration in comparison to the control group. At a higher concentration of lithium (>5 mM), we detected a toxicity effect of lithium, as was reported in the other systems. Therefore, lithium is able to support the regeneration of retinal axons under its therapeutically relevant concentrations.

### Example 15

### Lithium Supports RGC Survival in Culture

To determine whether lithium affects specifically the growth and survival of RGCs, we then developed a method to purify RGCs from the mouse retina, using antibody against a RGC marker, Thy1.2, conjugated with magnetic beads. Cell populations that reacted either positive or negative with anti-Thy1.2 were collected and seeded in culture, respectively. Our results showed that the procedure isolated nearly all of the RGCs of each retina.

To confirm that isolated cells were in fact RGCs, we pre-labeled RGCs by placing a retrograde tracer DiI in the SC one day before the procedure. Following isolation, the number of cells revealing cytoplasmic DiI was counted. We found that the isolated cell population consisted essentially of RGCs, as evidenced by the presence of cytoplasmic DiI. We further verified the purity of RGCs by detecting the expression of Thy1.2 antigen with immunofluorescence staining. We found that 90-95% of isolated cells were Thy1.2 positive. The isolated cells revealed similar morphology as RGCs described by Barres et al. (1988) that were purified with the antibody-panning methods. Therefore, we successfully purify the majority of RGCs with the magnetic-bead separation method.

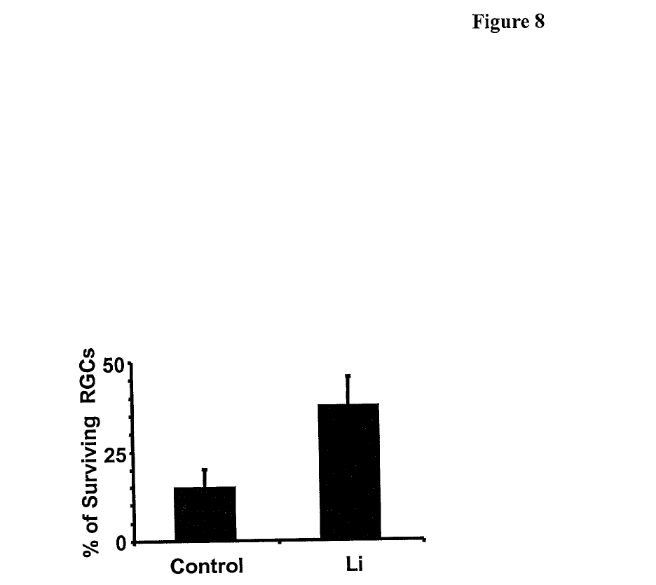

Using isolated RGC cultures, we investigated whether lithium acted directly on RGCs to promote their survival and axon regeneration. Purified RGCs were cultured in the absence and presence of lithium chloride (1 mM). After 5 days of incubation, RGC survival was assessed using LIVE/DEAD Cytotoxicity staining kit (Molecular probe). We found that in the absence of lithium, a majority of RGCs died within 5 days. Addition of lithium chloride promoted significantly the survival of RGCs up to three fold (FIG. 8). Most surviving RGCs revealed normal cell morphology and grew neurites. The result suggests that lithium can act on RGCs directly to exert neuroprotective and regenerative functions.

### Example 16

### Induction of Bcl-2 Expression in the Mouse Retina by Lithium

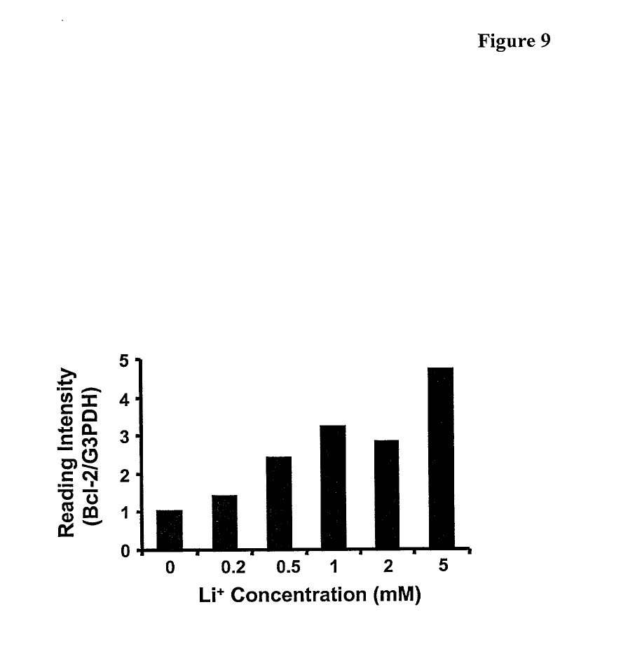

We next investigated whether lithium exerted its neuroprotective and regenerative effects via induction of Bcl-2 expression in the mouse retinas. Retino-brain slice co-cultures were prepared and incubated in the absence and presence of different concentrations of lithium chloride. Following five days of co-culturing, retinal RNAs were collected, and the levels of Bcl-2 expression were analyzed by quantitative RT-PCR. We found that when cultures were exposed to increasing amounts of lithium chloride, Bcl-2 mRNA levels of the retinas increased in parallel, in a dose dependent manner (FIG. 9). The up-regulation of Bcl-2 in the retinas could be observed when the concentration of lithium was as low as 0.2 mM and increased to ˜3 fold higher over that of control when it was 1 mM. Therefore, we observed a close correlation between the induction of Bcl-2 expression and promotion of retinal axon regeneration by lithium, implicating that induction of Bcl-2 expression is, at least, one of the mechanisms through which lithium mediates RGC functions.

### Example 17

### The Regeneration-Promoting Effect of Lithium is Bcl-2-Dependent

To further elucidate whether Bcl-2 is essentially involved in lithium-mediated RGC survival and axon regeneration, we studied genetically engineered mice deficient in Bcl-2 function (knockout) and mice over-expressed Bcl-2 transgene.

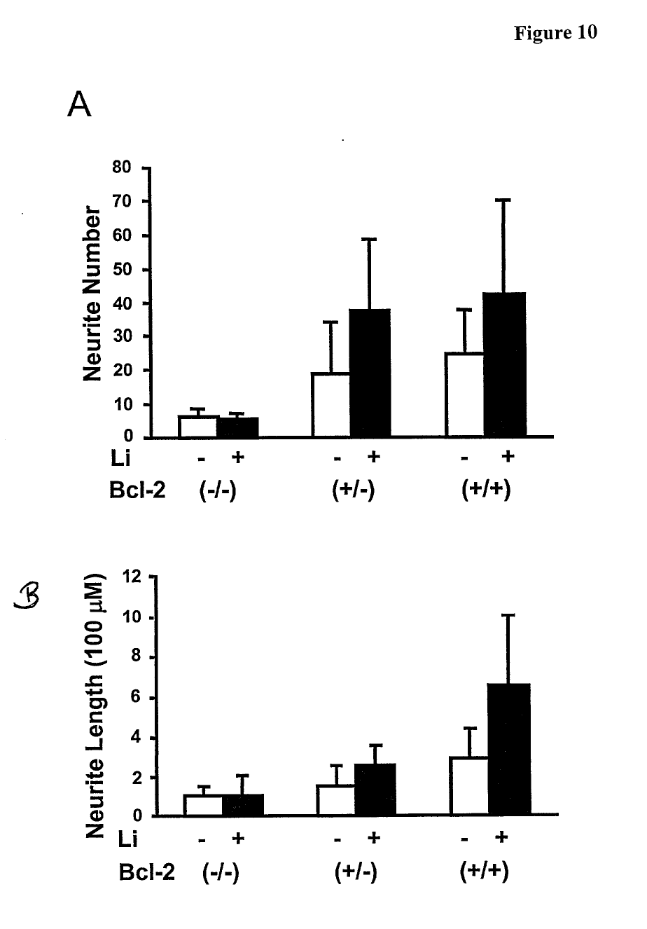

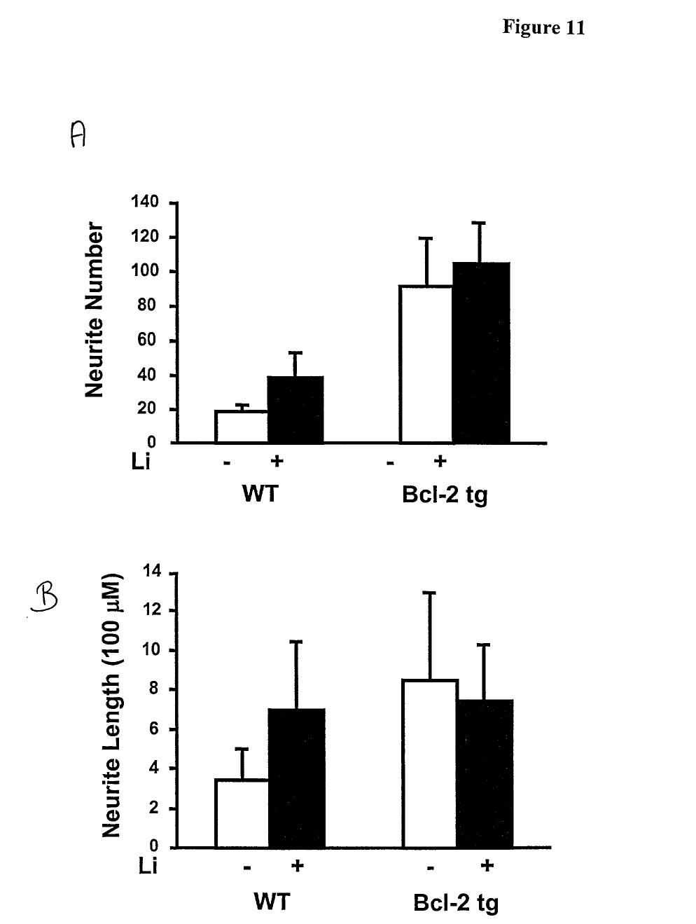

We first examined retino-brain slice co-cultures prepared from Bcl-2 knockout mice to determine whether Bcl-2 is required for lithium-induced RGC functions. Bcl-2 knockout mice were obtained from heterozygous breedings. Thus, within each littermate, only 25% of the mice would be Bcl-2 homozygous knockout; the other 50% would be heterozygous for Bcl-2 and 25% were wild-type. Co-cultures were prepared and scored blindly before mouse genotypes were determined, using a standard PCR methodology on mouse-tail DNA. In the absence of lithium, cultures derived from Bcl-2 knockout mice displayed much less vigorous neurite outgrowth from the retina into brain slices than those prepared from wild-type and heterozygous littermates (FIG. 10). Treatment with 1 mM lithium failed to promote retinal axon regeneration in cultures prepared from Bcl-2 knockout mice, while it induced 2-fold increase of axon regeneration in cultures prepared from both wild-type and heterozygous mice (FIG. 10). The result suggests that Bcl-2 is essentially involved in lithium induced retinal axon regeneration. To further investigate whether induction of Bcl-2 expression was the only factor contributing to the lithium-induced retinal axon regeneration, we then studied mice overexpressing Bcl-2. Bcl-2 transgenic mice were obtained from matings of wild-type C57BL/6J females with males carrying a Bcl-2 transgene under the control of neuron-specific enolase promoter (line 73a). Martinou J C, Dubois-Dauphin M, Staple J K, Rodriguez I, Frankowski H, Missotten M, Albertini P, Talabot D, Catsicas S, Pietra C (1994) Overexpression of BCL-2 in transgenic mice protects neurons from naturally occurring cell death and experimental ischemia. Neuron 13:1017-1030. Thus, within each littermate, half of the offspring would be Bcl-2 transgenic, while the other half would be wild-type, to serve as littermate controls. Retino-brain slice co-cultures were prepared before mouse genotypes were decoded. Consistent with our previous finding, co-cultures prepared from Bcl-2 transgenic mice exhibited robust retinal axon regeneration in comparison to those prepared from their littermate controls (FIG. 11). Overexpression of Bcl-2 stimulated more than 4-fold increase of axon regeneration from the retina into the brain slices over those of wild-type controls. Addition of lithium (1 mM) promoted retinal axon extension in cultures prepared from wild-type mice, but not in cultures from Bcl-2 overexpressing mice. Taken together, we conclude that lithium mediates RGC survival and retinal axon regeneration via induction of Bcl-2 expression.

These results for the first time suggest that lithium may be used as a therapeutic drug for treating retinal neurodegeneration, e.g., glaucoma, which involves both the optic nerve damage and RGC loss. It also offers new clues for better understanding of the regulation of retinal and CNS regeneration.

### Example 18

### Materials and Methods for Examples 14-17

Animals: Adult C57BL/6J mice, mice deficient in Bcl-2, and mice overexpressing Bcl-2 transgene driven under the neural specific enolase promoter were maintained in the mouse facility of the Schepens Eye Research Institute. Mouse genotypes were determined after sacrifice, using a standard polymerase chain reaction (PCR) methodology on tail DNA.

Retino-brain slice co-cultures: Retino-brain slice co-cultures were prepared as described above. Briefly, 2-day-old mouse pups were anesthetized by hypothermia and sacrificed. Their tails were collected, and a standard PCR procedure was used to determine mouse genotypes. The retinas and brains were dissected in HBSS. Coronal brain slices (300 μm) were prepared with a MacIIwain tissue chopper, and those contained the superior colliculus (SC) were selected and placed abut retinal explants in culture inserts. The co-cultures were maintained for 5 days in Neurobasal Medium (GIBCO) supplemented with B27 (GIBCO), 0.5 mM glutamine, and 12.5 μM glutamate. LiCl (1 mM; Sigma-Aldrich, cat. # L-4408) was added to the cultures at the day of plating. Cultures were then fixed with 4% paraformaldehyde. Retinal axons were labeled by placing 4 crystals of a fluorescent dye DiI into each retinal explant. After 2 weeks to allow dye diffusion, the cultures were visualized under the fluorescent microscope. The number of labeled axons that regenerated into the SC was quantified.

Isolation of RGCs: RGCs were isolated from 8-day-old mouse pups, using an antibody against a RGC specific marker, Thy1.2, conjugated with magnetic bead. Mouse pups were anesthetized by hypothermia, and their retinas were dissected in Mg²⁺/Ca²⁺ free Hank's balanced salt solution (HBSS). The retinas were incubated for 10 minutes, 37° C., in HBSS containing 1% papain and 5 U/ml DNase and transferred to a solution with papain inhibitor, ovomucoid (10%) or further dissociation and trituration. Dissociated cells were treated for 15 minutes at 4° C. with rabbit antibody against mouse Thy1.2 (CD90) conjugated with micro metal beads (Multinyi Biotech) in A buffer: phosphate buffered saline (PBS) with 0.5% Bovine Serum Albumin and 2 mM EDTA. In the presence of a strong magnetic field, cell suspensions were loaded onto a metal column. Cells that were labeled negative for Thy1.2 did not adhere to the column and were thus eluted with A buffer. Cells bound with anti-Thy1.2 were adhered to the column and were collected with A buffer after the removal of the magnetic field.

Characterization of the Isolated RGCs: To determine the purity of isolated RGCs, we performed both retrograde labeling and immunofluorescence staining assays. P0 mouse pups were anesthetized by hypothermia. Fluorescence tracer 1,1′-dioctadecyl-3,3,3′,3′-tetramethylindocarbocyanine perchlorate (DiI; Molecular probes, Eugene, Oreg.) (6% dissolved in dimethyformamide) was injected to the superior colliculus (SC) to cover whole areas. After 24 hr, mouse pups were anesthetized. Retinas were dissected and examined under a fluorescence microscope to ensure proper retrograde transport of DiI. After RGCs were isolated with magnetic bead separation and seeded in culture, percentage of cells with DiI labeling was recorded.

For immunofluorescent staining, isolated RGCs were seeded in culture and fixed with 4% paraformaldehyde. Briefly, cells were blocked for 15 min at room temperature in PBS containing 2.5% fetal bovine serum, 2.5% goat serum and 0.2% Triton X-100. Subsequently, they were incubated at room temperature for about 2 hours with primary antibody against Thy1.2, washed 3 times in PBS, and incubated for another 2 hr at room temperature with FITC-conjugated secondary antibody IgG (1:200, Chemicon). Immunofluorescence labeling was then observed under a Nikon TE300 microscope equipped with fluorescence illumination, and the number of immunofluorescent labeled cells in the isolated RGC population was counted.

Culture conditions and cell viability assay: 24-cell-well plates were pre-coated with 100 μg/ml Poly-D-Lysine (SIGMA) for 2 hr followed by 2 μg/ml human merosin (GIBCO) for 2 hr. Approximate 1×10⁵ purified RGCs were seeded onto each well. Cultures were maintained at 37° C. in humidified 5% CO₂ and 95% air in Neurobasal medium (GIBCO) with the addition of B27 (GIBCO), 100 U/ml penicillin-streptomycin, 0.5 mM glutamine, and 12.5 μM glutamate. Cultures were treated with different concentrations of LiCl (0.1-5 mM).

Cell viability was determined using a LIVE/DEAD cell cytotoxicity staining kit (Molecular probes). RGC cultures were incubated at room temperature for 45 minutes in PBS contained Calcein (10 μg/ml) and Ethidium D (5 μg/ml). Calcein is cleaved by live cells and yields cytoplasmic green fluorescence, while Ethidium D labels nucleic acids of dead cells with red fluorescence. The cultures were visualized and the number of live (green) and dead (red) cells were counted under an inverted Nikon TE300 microscope equipped with fluorescent illumination and phase contrast.

Semi-quantitative Reverse Transcription-Polymerase Chain Reaction (RT-PCR): Total retinal RNA was extracted with Trizol (GIBCO), according to the manufacturer's instructions. One microgram of total RNA was subjected to reverse transcription in a total volume of 20 μl reaction mixture that contained 4 μl reverse transcription buffer (5×), 1 μg of Oligo-dT primer, 0.5 mM each of the dNTPs (GIBCO), 10 mM Dithiothreitol, and 5 U of RNase inhibitor. The reaction was carried out under 42° C. for 45 minutes with 1U of Superscript II reverse transcriptase and terminated by incubating at 75° C. for 15 minutes.

Each PCR reaction contained equivalent amounts of cDNA. For relative quantitation as used in this study, the relative amount of target gene Bcl-2 in different samples was determined and compared with the amount of the internal standard control gene, G3PDH. PCR primers for detection of mouse Bcl-2 were designed to span an intron according to the Bcl-2 gene sequence so that the amplification of potentially contaminating genomic DNA would produce PCR fragments that were substantially larger than the cDNA PCR products. The DNA sequences of forward and reverse primers were as follows: Bcl-2 sense 5′-GCTGCAGACAGACTGGCCAG-3′ (SEQ ID NO: 5), antisense 5′-AGGCATCGCGCACATCCAGC-3′ (SEQ ID NO: 6); G3PDH sense 5′-CTGGAAGCCGGCGCAGATC-3′ (SEQ ID NO: 7), and antisense 5′GCGTGTCCAGGAAGCCTTCC-3′ (SEQ ID NO: 8).

PCR mixture consisted of 2.5 μl PCR buffer (10×), 1.5 mM Mg²⁺, 0.2 mM dNTP (GIBCO), primers, and 1U Taq DNA polymerase. PCR reactions were performed with GeneAmp PCR System 9700 (Perkin Elmer, Foster City, Calif.), run with the following program: 1 cycle of incubation at 94° C. for 4 minutes followed by 32 cycles of denaturating at 94° C., 1 minutes; annealing at 55° C., 30 seconds; extension at 72° C., 45 seconds. The reactions were ended with 1 cycle of a final extenstion at 72° C., 7 minutes. PCR products were resolved on 2% agarose gel electrophoresis and visualized with ethidium bromide stain and UV illumination.

Statistical Analysis: All data are expressed as mean±SD, and statistic significance, which was defined by p<0.05 was analyzed by Student t-test.

### Example 19

### Treatment of Glaucoma with Lithium in a Rat Glaucoma Model

This example describes methods for demonstrating the effect of lithium on glaucoma. A preferred glaucoma animal model is the rat animal model of chronic, moderately elevated intraocular pressure (IOP). This animal model is described, e.g., in Neufeld et al. (1999) PNAS 96: 9944. In this animal model, there is a slow optic nerve degeneration and loss of retinal ganglion cells that resembles glaucoma in humans. In this model, retinal ganglion cell degeneration is obtained by causing elevated IOP by cautery of one or more (e.g., three) episcleral vessels, as described in Neufeld et al., supra.

Adult male Wistar rats weighing about 250g will be used. Elevated IOP will be produced as described in the Neufeld et al., supra. One group of rats will be treated with Lithium Chloride (LiCl; obtained from Sigrna-Aldrich (cat. # L-4408)) in drinking water at a concentration of 15 mM for up to six months. A second group of rats will be used as untreated control. After six months of survival, the condition of the retinal ganglion cells and optic nerves of the rats will be evaluated. For this, Fluoro-Gold (Fluorochrome, Englewood, Colo.) will be injected into the superior colliculi of the rats. One week later, the rats will be sacrificed and flat-mount retinas will be prepared. Labeled retinal ganglion cells will be counted. The optic nerves will be cut into 1 μm cross-sections and stained for myelin. The densities of the optic nerve fibers will be recorded. The details of these procedures are set forth in Neufeld et al., supra.

The observation that animals treated with LiCl have more ganglion cells than the control rats will indicate that LiCl prevents retinal degeneration and can be used for treating or preventing glaucoma.

Another animal model of glaucoma that can be used is the monkey model of laser-induced glaucoma, as described, e.g., in Quigley et al. (1995) Invest. Ophtahalmol. Visual Sci. 36: 774.

Equivalents

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

1. A composition comprising lithium or a salt thereof and an agent that creates an environment favorable for axonal growth and a pharmaceutically acceptable carrier.

2. The composition of claim 1, wherein the agent is selected from the group consisting of NGF, BDNF, NT-3, 4, 5, or 6, CNTF, LIF, IGFI, IGFII, GDNF, GPA, bFGF, TGFB, and apolipoprotein E.

3. A vehicle for administration to a subject, comprising the composition of claim 1.

4. The vehicle of claim 3, which is a tube, catheter or stent.

5. The vehicle of claim 4, which is a syringe.

6. The composition of claim 1, which is in the form of a tablet.

7. A method for promoting axonal growth in a neural cell, comprising contacting the neural cell with an amount of lithium or salt thereof sufficient to stimulate axonal growth, such that axonal growth occurs.

8. The method of claim 7, wherein the neural cell is a central nervous system (CNS) neural cell.

9. The method of claim 7, wherein the neural cell is in the peripheral nervous system.

10. A method for treating a subject that has suffered a traumatic injury in which nerve cell injury has occurred, comprising administering to the subject lithium or a salt thereof, in an amount sufficient to stimulate axon regeneration, such that the subject is treated.

11. The method of claim 10, wherein administering comprises providing lithium or a salt thereof to the site of nerve cell injury.

12. The method of claim 11, wherein the lithium or a salt thereof is injected into the site of nerve cell injury.

13. The method of claim 10, further comprising administering an agent that creates an environment favorable for axonal growth.

14. The method of claim 13, wherein the agent is selected from the group consisting of NGF, BDNF, NT-3, 4, 5, or 6, CNTF, LIF, IGFI, IGFII, GDNF, GPA, bFGF, TGFB, and apolipoprotein E.

15. The method of claim 10, wherein the nerve cell injury is a spinal cord injury.

16. The method of claim 10, wherein the nerve cell injury is a peripheral nervous system injury.

17. The method of claim 10, wherein the nerve cell injury is an optic nerve injury.

18. A method for treating a subject for a state characterized by diminished potential axonal growth, comprising administering to the subject lithium or a salt thereof, in an amount sufficient to stimulate axonal growth, such that the subject is treated.

19. The method of claim 18, wherein the state is a CNS disorder.

20. The method of claim 18, wherein the state is a peripheral nervous system disorder.

21. The method of claim 18, wherein the state is glaucoma.

22. A method for stimulating axon growth of a neural cell in vitro, comprising contacting a neural cell with an amount of lithium or salt thereof sufficient to stimulate axon growth, such that the neural cell growths at least one axon.

23. The method of claim 22, wherein the neural cell is obtained from a subject.

24. The method of claim 22, wherein the neural cell is a cell that was differentiated from a stem cell.

25. A method for treating a subject for a state characterized by diminished potential axonal growth or a traumatic injury in which nerve cell injury has occurred, comprising administering to the subject a cell obtained according to the method of claim 22.

26. A method for treating a subject for a state characterized by diminished potential axonal growth or a traumatic injury in which nerve cell injury has occurred, comprising obtaining a neural cell from the subject, treating the cell according to the method of claim 22, and administering the neural cell with at least one axon back into the subject.

27. A method for preventing neural cell degeneration, comprising contacting the neural cell with an agent that increases the amount of Bcl-x_{L} in the neural cell, such that neural cell degeneration is prevented.

28. The method of claim 27, comprising contacting the neural cell with a nucleic acid encoding a Bcl-x_{L} protein or portion thereof sufficient for preventing neural cell degeneration.

29. The method of claim 27, comprising contacting the neural cell with a Bcl-x_{L} protein, such that the protein enters the neural cell.

30. A method for treating a neurodegenerative disease in a subject, comprising contacting neural cells of the subject that are undergoing neurodegeneration with an agent that increases the amount of Bcl-x_{L} in the neural cell, such that the neurodegenerative disease is treated in the subject.

31. A method for treating a subject having a partial or complete sectioning of the spinal cord or a nerve, comprising providing the ends of the spinal cord or nerve within less than about 100 μm distance from each other; and
contacting at least one cell from the spinal cord or nerve with an agent that increases the level of bcl-2 protein within the cell, such that the cell grows at least one axon, to thereby treat the subject.

32. The method of claim 31, wherein the agent is provided at the site of the sectioning of the spinal cord or nerve.

33. The method of claim 31, wherein the agent is lithium or a salt thereof.
